(19) United States
(12) Patent Application Publication
(10) Pub. No.: US 2004/0253307 A1
(43) Pub. Date: Dec. 16, 2004

(54) Sugar-free oral transmucosal solid dosage forms and uses thereof

(76) Inventors: Brian Hague, Bountiful, UT (US); Lynn J. Maland, Salt Lake City, UT (US)

Correspondence Address: CEPHALON, INC., 145 BRANDYWINE PARKWAY, WEST CHESTER, PA, 19380-4245, US

(21) Appl. No.: 10/771,046

(22) Filed: Feb. 3, 2004

Related U.S. Application Data
(60) Provisional application No. 60/444,832, filed on Feb. 4, 2003.

(51) Int. Cl. A61K 9/20

(52) U.S. Cl. 424/464

(57) ABSTRACT

The present invention is directed to oral solid dosage forms. The solid dosage forms are sugar-free, and comprise a pharmaceutical agent and a suitable pharmaceutically acceptable excipient. Preferably, the solid dosage forms of the present invention are bioequivalent to a sugar-containing solid dosage form. Bioequivalence is preferably obtained by incorporating an ionizing agent, more preferably in the form of a buffer system, into the solid dosage forms, in an amount sufficient to maintain a portion of the pharmaceutical agent, upon dissolution of said composition in saliva, in an ionized state.

### FIELD OF THE INVENTION

The present invention relates to oral drug delivery formulations. In particular, the invention relates to sugar-free solid pharmaceutical dosage forms for oral transmucosal delivery of pharmaceutically active substances.

### BACKGROUND OF THE INVENTION

Solid pharmaceutical dosage forms are well known in the art. Compared to other dosage forms, oral solid dosage forms are the most preferred dosage forms and account for eighty percent of all the pharmaceutical products on the market. Solid dosage forms are easier for a patient or caregiver to identify, handle and administer. They are also non-invasive and have high patient compliance.

Solid dosage forms can be further divided into several groups, based upon the route by which the drug is delivered, including, for example, gastrointestinal (GI) tract delivery, suppository (rectal, vaginal and urethral) delivery and oral transmucosal delivery. The majority of solid dosage forms on the market are designed for gastro-intestinal delivery. GI delivery is often referred to simply as “oral delivery,” because a tablet or capsule is initially introduced orally, and swallowed. However, this type of solid delivery form is designed to dissolve in the GI tract, where absorption of the drug occurs. Solids are also commonly delivered as suppositories such as laxatives, contraceptives and hemorrhoid medication. Relatively few drug formulations are designed as solid dosage forms intended to deliver a drug through the oral mucosa.

Despite the overall popularity of other delivery methods, oral transmucosal (OT) delivery is a particularly advantageous delivery route. One of the advantages of OT delivery is that it is non-invasive. Furthermore, OT delivery generally has better patient compliance, less risk of infection and lower cost than invasive procedures such as injection and implantation. It also has much shorter onset time, i.e., the time from administration to therapeutic effect, than does oral delivery. A drug absorbed via the oral mucosa will also avoid first pass metabolism, in which the drug is metabolized in the GI tract and liver. Oral transmucosal delivery is simple and can be administered by the caregiver or the patient with minimal discomfort.

The absorption of a drug across the mucosal tissue can be described using an expression based on Fick's law of diffusion:

$$\frac{ⅆ A}{ⅆ t} = \frac{{DK}_{p}}{h} \cdot ( {C}_{1} - {C}_{2} ) \cdot S$$

where dA is the amount of drug delivered over time dt, D is the diffusion coefficient of the drug inside the oral mucosal tissue, Kₚ is the partition coefficient of the drug between oral mucosal tissue and the drug solution, S is the surface area of the oral cavity, h is the thickness of the oral mucosal tissue, and C₁ and C₂ are the drug concentrations at the absorption site and in blood, respectively.

The capacity of oral transmucosal drug delivery is limited in large part by the surface area available for drug absorption. The surface area in the oral cavity is approximately 200 cm², which is relatively small compared to the surface area of other drug delivery routes, such as the GI tract (350,000 cm²) and skin (20,000 cm²).

The contact time between the drug and the absorption surface is primarily controlled by the dissolution rate of the solid unit. Once an immediate release solid unit is dissolved, any drug solution not yet absorbed will generally be swallowed shortly thereafter, thereby ending further OT drug absorption. Such immediate release dissolvable dosage units are usually designated as “open” delivery systems. A solid dosage unit can be designed to remain in the oral cavity for different periods of time. Generally, for rapid onset of effect, a solid unit is designed to remain in the oral cavity for about 10 to 15 minutes, when used as directed.

In addition to the difficulties presented by the oral cavity's unique environment, the physicochemical properties of the drug can present challenges and complications that affect oral transmucosal drug delivery. Primarily, the solubility, the dissolution rate, and the partition coefficient determine the extent to which a drug can be delivered via the oral mucosal tissue. Solubility of the drug and/or bulking agent can be a rate limiting step. Solubility and dissolution rate are key aspects in creating the concentration gradient, which is the driving force for drug delivery. Partition coefficient, on the other hand, acts like an amplifier, such that the drug delivery rate is directly proportional to the partition coefficient up to a point.

Various solid dosage forms have been used to deliver drugs via the oral mucosal tissue. U.S. Pat. No. 5,711,961 to Reiner, et al. discloses a chewing gum for the delivery of pharmaceuticals. The chewing gum delivery dosage form of Reiner is primarily directed to patients who may be more disposed to self-administer a drug in a chewing gum form as opposed to other less familiar dosage forms. The gum may also be used to mask the taste of various pharmaceutical ingredients. Reiner also discloses the use of the gum matrix in the formulation to extend the duration of drug delivery.

Transmucosal delivery of drugs may also be accomplished through the use of patches which are attached to the oral mucosa by a bio-adhesive. Oral transmucosal delivery using a buccal patch is disclosed in U.S. Pat. No. 5,298,256 to Flockhart, et al. The buccal patches are generally designed as a “closed” delivery system, that is, the environmental conditions inside the patch are primarily controlled by the formulation. Employing a closed delivery system can facilitate drug delivery, such as allowing the use of enhancers or other permeability facilitators in the formulation which might otherwise be impractical.

Solid dosage forms such as lozenges and tablets may also be used for oral transmucosal delivery of pharmaceuticals. For example, nitroglycerin sublingual tablets have been on the market for many years. The sublingual tablets are designed to deliver small amounts of the potent nitroglycerin, which is almost immediately dissolved and absorbed. On the other hand, most lozenges or tablets are typically designed to dissolve in the mouth over a period of at least several minutes which allows extended dissolution of the lozenge and absorption of the drug.

Administration of lozenges or sublingual tablets generally utilize an “open” delivery system, in which the drug delivery conditions are influenced by the conditions of the surrounding environment, such as rate of saliva secretion, pH of the saliva, or other conditions beyond the control of the formulation.

A lozenge-on-a-handle dosage form of transmucosal drug delivery is disclosed in U.S. Pat. No. 4,671,953 to Stanley, et al., the disclosure of which is incorporated herein by reference in its entirety for all purposes. In addition to being non-invasive and providing a particularly easy method of delivery, the lozenge-on-a-handle (or lozenge with an integrated oral transmucosal applicator) dosage form allows a patient or caregiver to move the dosage form in and out of the mouth to titrate the dose. This practice is called dose-to-effect, in which a patient or caregiver controls the administration of the dose until the expected therapeutic effect is achieved. This is particularly important for certain symptoms, such as pain, nausea, motion sickness, and premedication prior to anesthesia because each patient needs a different amount of medication to treat these symptoms. For these types of treatments, the patient is the only one who knows how much medication is enough. Once the appropriate amount of drug is delivered, the patient or caregiver can remove the lozenge-on-a-handle, thus, stopping delivery of the drug. This feature is especially important for particularly potent drugs, which may present a significant advantage of terminating drug administration once the desired effect is achieved.

Other oral transmucosal solid dosage forms are described in Stanley et al., U.S. Pat. Nos. 5,132,114; 5,288,497; 5,855,908; and 5,785,989; and U.S. Patent Application Publication No. 2002-0160043, the disclosures of which are incorporated herein by reference in their entirety, for all purposes. These references describe, inter alia, methods for producing solid dosage forms containing a drug in a dissolvable sugar-based matrix. The solid dosage forms may be produced, for example, by either mixing the drug into a molten sugar base, and allowing the base to solidify into a hard candy, or by compressing a powder, such as a compressible sugar, in which the drug has been dispersed, into a solid dosage form. Since these products contain large amounts of sugar, the bitterness or other unpleasant taste of the drug is masked. Additional flavoring enhancers or other sweeteners may also be included to provide an organoleptically satisfactory product.

A particularly successful example of the lozenge-on-a-handle-type oral transmucosal solid dosage form is the ACTIQ® brand of fentanyl citrate, that has been marketed in the United States and abroad for several years. In one ACTIQ® brand product, the active ingredient, fentanyl citrate, is intermixed in a sugar-based excipient EMDEX® (spray-crystallized maltose-dextrose magnesium stearate porous spheres), and compressed to produce what is essentially a drug-containing lozenge, to which a handle has been affixed. The product is approved for the control of break-through pain in opioid tolerant cancer patients. ACTIQ® is available in several strengths, and patients may regulate the amount of drug that is administered by varying the extent to which the product is rubbed over the oral mucosal surfaces, and the duration of administration. Through repeated usage, patients develop an understanding of just how much of the product will be needed to manage their pain. Since fentanyl is a very powerful narcotic, it was imperative for the manufacturers of the ACTIQ® product to assure consistency of the uniformity in the concentration of fentanyl and rate of dissolution of each dosage unit.

One characteristic of ACTIQ® and many other oral transmucosal solid dosage forms, however, is that the excipient used to prepare the matrix in which the drug is dispersed is sugar based. It would be desirable to provide sugar-free oral transmucosal solid dosage forms. Preferably, such dosage forms would fall within the FDA labeling requirements for classifying a product as sugar-free (i.e., less than 0.5 grams sugar per serving), so that consumers would be able to recognize that the product was suitable for use by diabetic patients, suitable for use by others seeking to avoid dietary sugar, and others seeking to avoid cariogenic dosage forms. However, such dosage forms must exhibit satisfactory dissolution rates that can be controlled by the patient, drug stability, and otherwise be suitable for oral transmucosal delivery, as discussed above. Moreover, where there already exists a population of users that depends upon a sugar-based product, and has grown accustomed to the predictable, patient-controlled rate of delivery needed to achieve the desired effect, it is important that such a sugar-free product exhibit a similar bioavailability and/or bioequivalence profile. The present invention is directed to solid dosage units embodying these attributes.

There is a definite need for sugar-free oral transmucosal solid dosage forms of fentanyl citrate which 1) lower overall sweetness for better patient acceptance, 2) reduce the incidence and/or potential for dental caries, 3) lower patient glycemic response, especially for Type II diabetics or patients with reduced glucose tolerance, 4) lower the caloric content for patients taking multiple dosages daily, and/or 5) provide a potential functional laxative effect as a benefit to the constipation often experienced as an adverse effect of opioid treatment. The present invention is directed to solid dosage units embodying these attributes.

### SUMMARY OF THE INVENTION

In view of the foregoing, the present invention is directed to sugar-free oral transmucosal solid dosage forms. In one embodiment, there is provided a pharmaceutical composition comprising an oral transmucosal solid dosage form that comprises a pharmaceutical agent, an ionizing agent, and a pharmaceutically acceptable excipient, wherein said composition is substantially sugar-free and is bioequivalent to a sugar-containing oral transmucosal solid dosage form, and wherein said ionizing agent is present in an amount sufficient to maintain a portion of said pharmaceutical agent, upon dissolution of said dosage form in saliva, in an ionized state.

Another embodiment of the invention is directed to a pharmaceutical composition comprising an oral transmucosal solid dosage form that comprises a pharmaceutical agent, a buffer, and a pharmaceutically acceptable excipient, wherein said composition is substantially sugar-free and is bioequivalent to a sugar-containing oral transmucosal solid dosage form, and wherein said buffer is present in an amount sufficient to maintain a portion of said pharmaceutical agent, upon dissolution of said dosage form in saliva, in an ionized state.

Another embodiment of the invention is directed to a pharmaceutical composition comprising an oral transmucosal solid dosage form that comprises an ionizable pharmaceutical agent, a buffer, and a pharmaceutically acceptable excipient, wherein said composition is substantially sugar-free and is bioequivalent to a sugar-containing oral transmucosal solid dosage form, and wherein said buffer is present in an amount sufficient to maintain a portion of said pharmaceutical agent, upon dissolution of said dosage form in saliva, in an ionized state.

Another embodiment of the invention is directed to a sugar-free pharmaceutical composition for the oral transmucosal delivery of fentanyl, said composition comprising fentanyl, or a pharmaceutically acceptable salt form thereof, and a pharmaceutically acceptable excipient, wherein said sugar-free composition is in the form of an oral transmucosal solid dosage form, and wherein said oral transmucosal solid dosage form is bioequivalent to a sugar-containing oral transmucosal solid dosage form containing fentanyl.

Another embodiment of the invention is directed to a sugar-free pharmaceutical composition for the oral transmucosal delivery of fentanyl citrate, said composition comprising fentanyl citrate a pharmaceutically acceptable excipient, wherein said sugar-free composition is in the form of an oral transmucosal solid dosage form, and wherein said oral transmucosal solid dosage form is bioequivalent to a sugar-containing oral transmucosal solid dosage form containing fentanyl citrate.

A further embodiment of the invention provides a method for the oral transmucosal delivery of a pharmaceutical agent in a sugar-free dosage form to a patient, comprising providing a composition comprising an oral transmucosal solid dosage form of the present invention, administering an effective amount of the composition to the oral mucosa of a patient, and delivering said pharmaceutical agent by absorption through a patient's oral mucosal tissue.

A further embodiment of the invention provides a method of treating pain which comprises introducing into the oral cavity of a patient a therapeutically effective amount of a sugar-free oral transmucosal solid dosage form according to the present invention wherein the ionizable pharmaceutical agent is fentanyl, or a pharmaceutically acceptable salt form thereof. In a preferred embodiment the pain is breakthrough pain, chronic pain, or migraine pain.

These and other objects and features of the present invention will become more fully apparent in the detailed description and appended claims to follow.

### DETAILED DESCRIPTION OF ILLUSTRATIVE EMBODIMENTS

The present application is directed to compositions and methods for administering same by oral transmucosal delivery. “Oral transmucosal delivery” refers to the delivery of a pharmaceutical agent across a mucous membrane in the oral cavity, pharyngeal cavity, or esophagus, and may be contrasted, for example, with traditional oral delivery, in which absorption of the drug occurs in the intestines. Accordingly, routes of administration in which the pharmaceutical agent is absorbed through the buccal, sublingual, gingival, pharyngeal, and/or esophageal mucosa are all encompassed within “oral transmucosal delivery,” as that term is used herein. Preferably, oral transmucosal delivery involves the administration of an oral transmucosal solid dosage form to the oral cavity of a patient, which is held in the oral cavity and dissolved, thereby releasing the pharmaceutical agent for oral transmucosal delivery. Of course, as the solid dosage form dissolves in the oral cavity, some of the saliva containing the pharmaceutical agent may be swallowed, and a portion of the drug may ultimately be absorbed from the intestines.

As used herein, the term “oral transmucosal solid dosage form” broadly refers to any solid delivery form suitable for administering a pharmaceutical agent by oral transmucosal delivery, including patches, troches, lozenges, pastilles, sachets, sublinqual tablets, lozenges-on-a-handle (otherwise referred to as lollipops), and the like. A preferred form includes patches, lozenges, sublinqual tablets, and lozenges-on-a-handle. An especially preferred form is the lozenge-on-a-handle, in which the solid dosage form has a handle affixed thereto. The solid dosage form may be held between the cheek and gum or placed on or under the tongue, or it may be actively licked, sucked, or rubbed across the oral mucosa by the patient or a caregiver. Preferably, the solid dosage form is not bitten or chewed, unless the broken pieces are then held in the mouth until substantially dissolved.

Employing the pharmaceutical compositions of the present invention, a pharmaceutical agent may be introduced into the patient's bloodstream almost as fast as through injection, and much faster than using the oral administration route, while avoiding the negative aspects of those delivery methods. The present invention achieves these advantages by incorporating the drug into a dissolvable matrix material. A solid dosage form within the scope of the present invention can be used to administer drugs in a dose-to-effect manner, or until the precise desired effect is achieved. In preferred embodiments, the dosage form has an appliance or handle attached thereto to permit easy removal from the patient's mouth, once the desired effect has been achieved.

Unlike the oral transmucosal solid dosage forms of the prior art, the solid dosage forms of the present invention are sugar-free. Sugar containing solid dosage forms generally contain large amounts of sugar, or mixtures of sugars, for example greater than about 50%, or even greater than about 90%. As used herein, the term “sugar” refers to mono-, di-, and oligo-saccharides, also known in the art as non-hydrogenated carbohydrates of empirical formula (CH₂O)ₙ, examples of which include glucose, mannose, galactose, ribose, dextrose, fructose, maltose, sucrose, levulose, and lactose. The term “sugar” notably includes saccharides that, when administered by oral transmucosal delivery, may be cariogenic and/or may be metabolized (for example, by hydrolysis or fermentation) to compounds that are cariogenic. For purposes of this application, the terms “glucose” and “dextrose” may be used interchangeably. The term “sugar” does not include polyhydric alcohols (sometimes referred to as “sugar alcohols” or hydrogenated saccharides), such as sorbitol, mannitol, xylitol, and erythritol, or the sugar derivatives of polyhydric alcohols, such as maltitol, lactitol, isomalt, and polyalditol. The term “sugar” also does not include complex carbohydrates such as gums and polysaccharides, including starch and cellulose, nor their derivatives, such as hydroxy ethyl starch and carboxymethylcellulose. Preferably, the term “sugar” also does not include mono-, di-, and oligosaccharides that are non-cariogenic.

The term “sugar-free” refers to compositions that are mostly free of “sugar,” as defined above. By “mostly free” it is meant that the compositions contain less than about 40.0% by weight sugar, as that term is defined above. Preferably, the compositions contain less than about 25% by weight, more preferably less than about 10% by weight sugar, on a dry weight basis. In a more preferred embodiment, the term “sugar-free” refers to compositions that are substantially free of “sugar,” as defined above. By “substantially free” it is meant that the compositions contain less than about 5.0% by weight sugar, as that term is defined above. Preferably, the compositions contain less than about 3% by weight, more preferably less than about 2% by weight, and even more preferably less than about 1% by weight sugar, on a dry weight basis. The sugar-free compositions of the present invention are also substantially free (as defined above) of complex carbohydrates and/or polysaccharides that may be readily converted to sugars in the oral cavity, when the solid dosage form is administered to a patient for oral transmucosal delivery.

As noted previously, however, it is important that an oral transmucosal solid dosage form exhibit satisfactory patient-controlled dissolution rates, drug stability, and otherwise be suitable for oral transmucosal delivery. In order to meet these criteria, it is generally necessary to disperse the pharmaceutical agent in a pharmaceutically acceptable excipient, that provides both bulk, and helps control the dissolution rate. The term “pharmaceutically acceptable,” as used herein, refers to materials which are generally not toxic or injurious to a patient when used in the compositions of the present invention, including when the compositions are administered by the oral transmucosal route, according to methods described herein. The term “patient,” as used herein, refers to animals, including mammals, preferably humans.

Preferably, the excipient is one that will not impart an unpleasant taste to the solid dosage form, such that it might deter a patient from using the product for oral transmucosal delivery. One type of pharmaceutically acceptable excipient that is particularly suitable for use in the present compositions, since it meets this requirement, is the class of excipient known as polyhydric alcohols. In particular the polyhydric alcohols that are commonly used in preparing sugar-free candies, are preferred. Exemplary polyhydric alcohols include, for example, but are not limited to, sorbitol, mannitol, xylitol, erythritol, maltitol, lactitol, isomalt, and polyalditol; and any of the optical isomers and crystalline forms of such polyhydric alcohols as may be used as an appropriate sugar-free substitute. Preferred polyhydric alcohols include xylitol, isomalt, and polyalditol. Various excipients containing these polyhydric alcohols are available commercially, and are widely known to those of ordinary skill in the art. In addition to their natural sweetness, these excipients are particularly suitable because they are non-cariogenic. In addition, when consumed in accordance with the methods of the present invention, they preferably do not lead to an increase in blood glucose, that may be contraindicated, for example, in diabetic patients. These polyhydric alcohols also preferably act as reduced calorie substitutes. Moreover, the use of these polyhydric alcohols for the preparation of sugar-free candies, lozenges, and other solid dosage forms is well known, such that those of skill in the art may easily use such excipients to prepare the oral transmucosal solid dosage forms of the present invention.

In addition to the polyhydric alcohol excipients discussed previously, the pharmaceutical compositions of the present invention may also contain other bulking agents and/or binding agents, including polymeric compounds, complex carbohydrates and their derivatives, and other materials that are well known to those of skill in the art, provided that the oral transmucosal solid dosage form still meet the definition of “sugar-free” provided previously. Examples of other bulking and/or binding agents include, but are not limited to, polydextrose, cellulosic ethers, and polyethylene glycols (PEG). Preferred examples of cellulosic ethers include, but are not limited to hydroxypropyl cellulose, hydroxyethyl cellulose, carboxymethylcellulose, and hydroxypropylmethyl cellulose, and derivatives and/or salt forms thereof. Polyethylene glycols are commercially available by grades of average molecular weight; preferred examples of polyethylene glycol include, PEG 3350 to PEG 20,000, more preferably, PEG 4000 to PEG 8,000; specifically, PEG 3350, PEG 4000 and PEG 8000. Frequently, commercially available excipients contain a mix of bulking agents. For example, a commercially available excipient such as XYLITAB® 200, which consists of xylitol plus 1.5% of the bulking agent carboxymethylcellulose, may be used as the excipient.

Suitable excipients for use in the present invention also include non-cariogenic mono-, di-, oligo-, or poly-saccharides. The term “non-cariogenic mono-, di-, oligo-, or poly-saccharides” refers to saccharide compounds that, when administered by oral transmucosal delivery, are not, or are only minimally metabolized to form acids in the mouth that may lead to the formation of dental caries (i.e., are non-cariogenic). The term “minimally metabolized to form acids in the mouth” means that less that about 10%, and more preferably less than about 5% of the non-cariogenic saccharide compounds may be metabolized, when administered by oral transmucosal delivery, to form acids that may lead to the formation of dental caries. An example of a non-cariogenic poly-saccharide is polydextrose.

The pharmaceutical compositions of the present invention also contain a pharmaceutical agent. “Pharmaceutical agent” refers to a substance which may be used in connection with an application that is therapeutic or diagnostic in nature, such as in methods for diagnosing the presence or absence of a disease in a patient and/or in methods for the treatment of disease in a patient. As used herein, “pharmaceutical agent” refers also to a substance which is capable of exerting a biological effect in vivo. The pharmaceutical agents may be neutral or positively or negatively charged. Examples of suitable pharmaceutical agents include, inter a/ia, diagnostic agents, pharmaceuticals, drugs, synthetic organic molecules, proteins, peptides, vitamins, and steroids.

Preferably, the pharmaceutical agents are “ionizable,” in that they contain at least one ionizable functional group. Ionizable functional groups can be acidic groups, or basic groups, with “acidic” and “basic” referring to acidic or basic behavior in a Brønsted-Lowry or Lewis acid/base sense. Acidic functional groups are those groups that can be deprotonated by a suitable base to yield the corresponding anionic group (the conjugate base), or groups that can accept an electron pair. Basic functional groups are those groups that can be protonated by a suitable acid to yield the corresponding cationic group (the conjugate acid), or can donate an electron pair. Of course, many suitable pharmaceutical agents contain a plurality of ionizable functional groups, and a single pharmaceutical agent may contain one or more acidic functional groups as well as one or more basic functional groups (e.g. a zwiterion). Such pharmaceutical agents are also within the scope of the present invention.

Acidic functional groups include, but are not limited to, carboxylic acids, imidazolidinediones, thiazolidinediones, pyrimidinetriones, hydroxyheteroaromatics, phenols, phosphoric acids, sulfuric acids, sulfonic acids, sulfonamides, aminosulfones, sulfonylureas, tetrazoles and thiols. In order to avoid particularly cumbersome terminology, the functional groups, whether acidic or basic, are referred to by naming the corresponding free compound. For example, referring to a functional group, the term “aminosulfone” is used, rather than the more technically precise term “aminosulfonyl.” This usage is common in the art, and is well understood by one skilled in the art.

Basic functional groups include, but are not limited to, aliphatic amines, aromatic amines, C-substituted aromatic amines, N-substituted aromatic amines, heterocyclic amines, C-substituted heterocyclic amines and N-substituted heterocyclic amines. Examples of aromatic amines and substituted aromatic amines include, but are not limited to, aniline, N-methylaniline and p-toluidine. Examples of heterocyclic and substituted heterocyclic amines include, but are not limited to, pyrrole, pyrazole, imidazole, indole, pyridine, pyridazine, pyrimidine, quinoline, piperidine, pyrrolidine, morpholine, thiazole, purine and triazole.

Specific examples of ionizable pharmaceutical agents having at least one ionizable acidic functional group include, but are not limited to: acetazolamide, acetohexamide, acrivastine, alatrofloxacin, albuterol, alclofenac, aloxiprin, alprostadil, amodiaquine, amphotericin, amylobarbital, aspirin, atorvastatin, atovaquone, baclofen, barbital, benazepril, bezafibrate, bromfenac, bumetamide, butobarbital, candesartan, capsaicin, captopril, cefazolin, celecoxib, cephadrine, cephalexin, cerivastatin, cetrizine, chlorambucil, chlorothiazide, chlorpropamide, chlorthalidone, cinoxacin, ciprofloxacin, clinofibrate, cloxacillin, cromoglicate, cromolyn, dantrolene, dichlorophen, diclofenac, dicloxacillin, dicumarol, diflunisal, dimenhydrinate, divalproex, docusate, dronabinol, enoximone, enalapril, enoxacin, enrofloxacin, epalrestat, eposartan, essential fatty acids, estramustine, ethacrynic acid, ethotoin, etodolac, etoposide, fenbufen, fenoprofen, fexofenadine, fluconazole, flurbiprofen, fluvastatin, fosinopril, fosphenyloin, fumagillin, furosemide, gabapentin, gemfibrozil, gliclazide, glipizide, glybenclamide, glyburide, glimepiride, grepafloxacin, ibufenac, ibuprofen, imipenem, indomethacin, irbesartan, isotretinoin, ketoprofen, ketorolac, lamotrigine, levofloxacin, lisinopril, lomefloxacin, losartan, lovastatin, meclofenamic acid, mefenamic acid, mesalamine, methotrexate, metolazone, montelukast, nalidixic acid, naproxen, natamycin, nimesulide, nitrofurantoin, non-essential fatty acids, norfloxacin, nystatin, ofloxacin, oxacillin, oxaprozin, oxyphenbutazone, penicillins, pentobarbital, perfloxacin, phenobarbital, phenyloin, pioglitazone, piroxicam, pramipexol, pranlukast, pravastatin, probenecid, probucol, propofol, propylthiouracil, quinapril, rabeprazole, repaglinide, rifampin, rifapentine, sparfloxacin, sulfabenzamide, sulfacetamide, sulfadiazine, sulfadoxine, sulfamerazine, sulfamethoxazole, sulfafurazole, sulfapyridine, sulfasalazine, sulindac, sulphasalazine, sulthiame, telmisartan, teniposide, terbutaline, tetrahydrocannabinol, tirofiban, tolazamide, tolbutamide, tolcapone, tolmetin, tretinoin, troglitazone, trovafloxacin, undecenoic acid, ursodeoxycholic acid, valproic acid, valsartan, vancomycin, verteporfin, vigabatrin, vitamin K-S (II) and zafirlukast.

Specific examples of suitable ionizable pharmaceutical agents having at least one ionizable basic functional group include, but are not limited to: abacavir, acebutolol, acrivastine, alatrofloxacin, albuterol, albendazole, alfentanil, alprazolam, alprenolol, amantadine, amiloride, aminoglutethimide, amiodarone, amitriptyline, amlodipine, amodiaquine, amoxapine, amphetamine, amphotericin, amprenavir, amrinone, amsacrine, apomorphine, astemizole, atenolol, atropine, azathioprine, azelastine, azithromycin, baclofen, benethamine, benidipine, benzhexol, benznidazole, benztropine, biperiden, bisacodyl, bisanthrene, bromazepam, bromocriptine, bromperidol, brompheniramine, brotizolam, bupropion, butenafine, butoconazole, cambendazole, camptothecin, carbinoxamine, cephadrine, cephalexin, cetrizine, cinnarizine, chlorambucil, chlorpheniramine, chlorproguanil, chlordiazepoxide, chlorpromazine, chlorprothixene, chloroquine, cimetidine, ciprofloxacin, cisapride, citalopram, clarithromycin, clemastine, clemizole, clenbuterol, clofazimine, clomiphene, clonazepam, clopidogrel, clozapine, clotiazepam, clotrimazole, codeine, cyclizine, cyproheptadine, dacarbazine, darodipine, decoquinate, delavirdine, demeclo-cycline, dexamphetamine, dexchlorpheniramine, dexfenfluramine, diamorphine, diazepam, diethylpropion, dihydrocodeine, dihydroergotamine, diltiazem, dimenhydrinate, diphenhydramine, diphenoxylate, diphenyl-imidazole, diphenylpyraline, dipyridamole, dirithromycin, disopyramide, dolasetron, domperidone, donepezil, doxazosin, doxycycline, droperidol, econazole, efavirenz, ellipticine, enalapril, enoxacin, enrofloxacin, eperisone, ephedrine, ergotamine, erythromycin, ethambutol, ethionamide, ethopropazine, etoperidone, famotidine, felodipine, fenbendazole, fenfluramine, fenoldopam, fentanyl, fexofenadine, flecainide, flucytosine, flunarizine, flunitrazepam, fluopromazine, fluoxetine, fluphenthixol, fluphenthixol decanoate, fluphenazine, fluphenazine decanoate, flurazepam, flurithromycin, frovatriptan, gabapentin, granisetron, grepafloxacin, guanabenz, halofantrine, haloperidol, hyoscyamine, imipenem, indinavir, irinotecan, isoxazole, isradipine, itraconazole, ketoconazole, ketotifen, labetalol, lamivudine, lanosprazole, leflunomide, levofloxacin, lisinopril, lomefloxacin, loperamide, loratadine, lorazepam, lormetazepam, lysuride, mepacrine, maprotiline, mazindol, mebendazole, meclizine, medazepam, mefloquine, melonicam, meptazinol, mercaptopurine, mesalamine, mesoridazine, metformin, methadone, methaqualone, methylphenidate, methylphenobarbital, methysergide, metoclopramide, metoprolol, metronidazole, mianserin, miconazole, midazolam, miglitol, minoxidil, mitomycins, mitoxantrone, modafinil, molindone, montelukast, morphine, moxifloxacin, nadolol, nalbuphine, naratriptan, natamycin, nefazodone, nelfinavir, nevirapine, nicardipine, nicotine, nifedipine, nimodipine, nimorazole, nisoldipine, nitrazepam, nitrofurazone, nizatidine, norfloxacin, nortriptyline, nystatin, ofloxacin, olanzapine, omeprazole, ondansetron, omidazole, oxamniquine, oxantel, oxatomide, oxazepam, oxfendazole, oxiconazole, oxprenolol, oxybutynin, oxyphencyclimine, paroxetine, pentazocine, pentoxifylline, perchlorperazine, perfloxacin, perphenazine, phenbenzamine, pheniramine, phenoxybenzamine, phentermine, physostigmine, pimozide, pindolol, pizotifen, pramipexol, pranlukast, praziquantel, prazosin, procarbazine, prochlorperazine, proguanil, propranolol, pseudoephedrine, pyrantel, pyrimethamine, quetiapine, quinidine, quinine, raloxifene, ranitidine, remifentanil, repaglinide, reserpine, ricobendazole, rifabutin, rifampin, rifapentine, rimantadine, risperidone, ritonavir, rizatriptan, ropinirole, rosiglitazone, roxatidine, roxithromycin, salbutamol, saquinavir, selegiline, sertraline, sibutramine, sildenafil, sparfloxacin, spiramycins, stavudine, sufentanil, sulconazole, sulphasalazine, sulpiride, sumatriptan, tacrine, tamoxifen, tamsulosin, temazepam, terazosin, terbinafine, terbutaline, terconazole, terfenadine, tetramisole, thiabendazole, thioguanine, thioridazine, tiagabine, ticlopidine, timolol, tinidazole, tioconazole, tirofiban, tizanidine, tolterodine, topotecan, toremifene, tramadol, trazodone, triamterene, triazolam, trifluoperazine, trimethoprim, trimipramine, tromethamine, tropicamide, trovafloxacin, vancomycin, venlafaxine, vigabatrin, vinblastine, vincristine, vinorelbine, vitamin K₅, vitamin K₆, vitamin K₇, zafirlukast, zolmitriptan, zolpidem and zopiclone.

The present invention has particular applicability to a variety of drugs affecting the central nervous system. For example, the present invention may easily be utilized in the administration of opioid agonists (such as fentanyl, alfentanil, sufentanil, lofentanil, and carfentanil), opioid antagonists (such as naloxone and nalbuphine), butyrophenones (such as droperidol and haloperidol); benzodiazepines (such as valium, midazolam, triazolam, oxazolam, and lorazepam); GABA stimulators (such as etomidate); barbiturates (such as Thiopental, methohexital, thiamazol, pentobarbital, and hexabarbital); di-isopropylphenols drugs (such as diprivan); and other central nervous system-acting drugs such as levodopa. It will be appreciated that other drugs may also be utilized within the scope of the present invention either singly or in combination. Table 1 lists some of the CNS-acting drugs which may be suitable for incorporation into the dosage form of the present invention, as well as some of the characteristics of those drugs.

TABLE 1

| GENERIC DRUG | DRUG CLASS | DOSE RANGE | |
|---|---|---|---|
| methohexital | barbiturate | 10-500 | mg |
| pentobarbital | barbiturate | 50-200 | mg |
| thiamylal | barbiturate | 10-500 | mg |
| thiopental | barbiturate | 50-500 | mg |
| fentanyl | opioid agonist | 0.05-5 | mg |
| alfentanil | opioid agonist | 0.5-50 | mg |
| sufentanil | opioid agonist | 5-500 | μg |
| lofentanil | opioid agonist | 0.1-100 | μg |
| carfentanil | opioid agonist | 0.2-100 | μg |
| nalbuphine | opioid agonist | 1-50 | mg |
| naloxone | opioid antagonist | 0.05-5 | mg |
| diazepam | benzodiazepine | 1-40 | mg |
| lorazepam | benzodiazepine | 1-4 | mg |
| midazolam | benzodiazepine | 0.5-25 | mg |
| oxazepam | benzodiazepine | 5-40 | mg |
| triazolam | benzodiazepine | 250-1000 | mg |
| droperidol | butyrophenone | 1-20 | mg |
| haloperidol | butyrophenone | 0.5-10 | mg |
| propanidid | substituted eugenol anesth. | 1-10 | mg |
| etomidate | GABA stimulator | 5-60 | mg |
| propofol | substituted phenol | 3-50 | mg |
| ketamine | phencyclidine | 5-300 | mg |

Drugs having effects on the cardiovascular and renal vascular systems may also be administered using a dosage form of the present invention. A few examples of such drugs are identified in Table 2.

TABLE 2

| GENERIC DRUG | DRUG CLASS | DOSE RANGE | |
|---|---|---|---|
| bretylium | antiarrhythmic | 50-500 | mg |
| captopril | ACE inhibitor | 25-75 | mg |
| clonidine | antihypertensive | 0.1-0.5 | mg |
| dopamine | renal vascular | 0.5-5 | mg |
| enalapril | ACE inhibitor | 5-15 | mg |
| esmolol | antihypertensive/angina | 100-250 | mg |
| furosemide | diuretic | 20-100 | mg |
| isosorbide | angina | 2.5-40 | mg |
| labetolol | antihypertensive | 100-400 | mg |
| lidocaine | antiarrhythmic | 50-250 | mg |
| metolazone | diuretic | 5-50 | mg |
| metoprolol | antihypertensive | 25-100 | mg |
| nadolol | antihypertensive | 40-160 | mg |
| nifedipine | antihypertensive/angina/vasodilator | 10-40 | mg |
| nitroglycerin | antihypertensive/angina | 0.4-1.0 | mg |
| nitroprusside | hypotensive | 10-50 | mg |
| propranolol | antihypertensive/angina | 0.1-50 | mg |

In addition to the foregoing, there are many other drugs which can be administered using a dosage form of the present invention. Exemplary of such drugs are those identified in Table 3.

TABLE 3

| GENERIC DRUG | DRUG CLASS | DOSE RANGE | |
|---|---|---|---|
| benzquinamide | antiemetic | 25-100 | mg |
| meclizine | antiemetic | 25-100 | mg |
| metoclopramide | antiemetic | 5-20 | mg |
| prochlorperazine | antiemetic | 5-25 | mg |
| trimethobenzamide | antiemetic | 100-2500 | mg |
| clotrimazole | antifungal | 10-20 | mg |
| nystatin | antifungal | 1-5 × 10⁶ | units |
| carbidopa | antiparkinson with levodopa | 10-50 | mg |
| levodopa | antiparkinson | 100-750 | mg |
| sucralfate | gi protectant | 1-2 | grams |
| albuterol | bronchodilator | 0.8-1.6 | mg |
| aminophylline | bronchodilator | 100-500 | mg |
| beclomethasone | bronchodilator | 20-50 | μg |
| dyphylline | bronchodilator | 100-400 | mg |
| epinephrine | bronchodilator | 200-500 | μg |
| flunisolide | bronchodilator | 25-50 | μg |
| isoetharine | bronchodilator | 170-680 | μg |
| isoproterenol hcl | bronchodilator | 60-260 | μg |
| metaproterenol | bronchodilator | 0.65-10 | mg |
| oxtriphylline | bronchodilator | 50-400 | mg |
| terbutaline | bronchodilator | 2.5-10 | mg |
| theophylline | bronchodilator | 50-400 | mg |
| ergotamine | antimigraine | 2-4 | mg |
| methysergide | antimigraine | 2-4 | mg |
| propranolol | antimigraine | 80-160 | mg |
| suloctidil | antimigraine | 200-300 | mg |
| ergonovine | oxytocic | 0.2-0.6 | mg |
| oxytocin | oxytocic | 5-20 | units |
| desmopressin | antidiuretic | 10-50 | μg |
| acetate | | | |
| lypressin | antidiuretic | 7-14 | μg |
| vasopressin | antidiuretic | 2.5-60 | units |
| insulin | antihyperglycemic | 1-100 | units |

In addition to the foregoing drugs, certain macromolecular drugs (such as β-endorphin, enkephalins, bradykinin, angiotensin 1, gonadotropic hormones, adrenocorticotropic hormone (ACTH), calcitonin, parathyroid hormone, and growth hormone), polysaccharides (such as heparin and low molecular weight heparin), antigens, antibodies, and enzymes may be adapted for transmucosal administration within the scope of the present invention.

When incorporating a drug into a dissolvable matrix within the scope of the present invention, the amount of drug used will generally differ from the amount used in more traditional injection and oral administration techniques. Depending upon the lipophilic nature of the drug, its potency, solubility, the use of permeation enhancers, and the drug's end use, the total concentration of the drug in the typical dosage form may contain up to 50 times more than the amount of drug that would typically be used in an injection, but it may also contain significantly less than the amount used orally, and it may also contain less than the amount used in some intramuscular injections. For purposes of example, Tables 1, 2, and 3 set forth presently contemplated ranges of the dosages of certain drugs which could be typically used.

As noted previously, however, patients using prior art sugar-based oral transmucosal solid dosage forms may have already become accustomed to the rate of onset, and the drug effect that may be achieved from using such products. Where the drug exerts a potent effect on the central nervous system, for example, it may be vital that the sugar-free solid dosage form have a bioavailability and/or bioequivalence profile that is similar to a sugar-containing solid dosage form, to which they may have become accustomed, lest over-dosage result. Thus, it is preferred that a sugar-free oral transmucosal solid dosage form of the present invention be bioequivalent to a sugar-containing oral transmucosal solid dosage form. “Bioequivalent”, as used herein, refers to the standard applied by the respective national regulatory agency in a country for which marketing approval of the invention is sought. For example, for a composition of the invention to be bioequivalent in the United States, a composition of the invention must comply with the definition of bioequivalence as defined by the U.S. Food and Drug Adminstration in 21 CFR 320.1. Similarly, two solid dosage forms are considered bioequivalent, as that term is used herein, if the rate and extent of absorption of the pharmaceutical agent present in the dosage forms are not significantly different, when administered to patients or subjects at the same molar dose under similar experimental conditions.

Methods for assessing the rate and extent of absorption of a pharmaceutical agent, including availability of the pharmaceutical agent at the site of drug action, are well known to those of skill in the art. Typical methods involve measuring the drug concentration in the blood at various points of time after administration of the drug, and then integrating the values obtained over time, to yield the total area under the drug-concentration vs. time curve (AUC). The AUC measurement is a direct measurement of the bioavailability of the drug. This assessment also allows one to determine the maximum drug concentration that is achieved in the blood following administration (i.e., the Cₘₐₓ), and the average time to achieve that maximum concentration (i.e., the tₘₐₓ). The rate and extent of absorption of the pharmaceutical agent in two solid dosage forms are considered to be “not significantly different,” and the two forms considered to be bioequivalent, if the mean ratios comparing the pharmacokinetic parameters Cₘₐₓ and AUC for two products, and, more preferably, Cₘₐₓ, AUC, and tₘₐₓ, for two products fall within 0.8 to 1.25, respectively, with a 90% Confidence Interval (CI).

One method of determining whether a pharmaceutical composition of the invention is bioequivalent to a sugar-containing oral transmucosal solid dosage form is to compare the pharmacokinetic parameters Cₘₐₓ, AUC, and tₘₐₓ, of the pharmaceutical composition of the invention to the Cₘₐₓ, AUC, and tₘₐₓ of the approved commercial product of the sugar-containing oral transmucosal solid dosage form, data, for which, is publicly provided in the product package insert. For example, the package insert for ACTIQ® (a sugar-containing oral transmucosal solid dosage form of fentanyl citrate) discloses the pharmacokinetic parameters Cₘₐₓ, AUC, and tₘₐₓ for four therapeutic unit dosages. A list of the approved dosages, together with their pharmacokinetic parameters Cₘₐₓ, AUC, and tₘₐₓ, is shown in Table 4, below.

TABLE 4

| pharmacokinetic parameters | 200 μg | 400 μg | 800 μg | 1600 μg |
|---|---|---|---|---|
| Cₘₐₓ (ng/ml) | 0.39 | 0.75 | 1.55 | 2.51 |
| AUC (ng/ml min) | 102 | 243 | 573 | 1026 |
| tₘₐₓ (min) | 40 | 25 | 25 | 20 |

Thus, a pharmaceutical composition of the invention containing fentanyl citrate would be considered bioequivalent to any single dosage form of ACTIQ® if the ratio of the respective pharmacokinetic parameters were within 0.8 to 1.25, with a 90% Confidence Interval, when administered to patients or subjects under similar experimental conditions.

It has been found, however, that sugar-free oral transmucosal solid dosage forms are not bioequivalent to oral transmucosal solid dosage forms that contain sugar. This may result from a variety of physical and chemical factors present in the different formulations. For example, in one study comparing a sucrose-glucose oral transmucosal fentanyl citrate solid dosage form with a sorbitol-based (i.e., sugar-free) oral transmucosal fentanyl citrate solid dosage form of equal strength, it was determined that the Cₘₐₓ and AUC were much higher for the sugar-free solid dosage form, and tₘₐₓ was significantly shorter for the sugar-free formulation. See Lind, G H, et al., “Fentanyl absorption is influenced by the sugar matrix formulation of oral transmucosal fentanyl citrate,” Anesthesiology, 38, September, 1995 (Abstract 299). Thus, the two formulations were not bioequivalent, and substitution of the sugar-free solid dosage form for the one containing sugars would be accompanied by risks of an overdosage.

As discussed previously, a variety of factors influence the rate of absorption, and hence the bioavailability, of a pharmaceutical agent from an oral transmucosal solid dosage form. Accordingly, various methods may be used to adjust the formulation to achieve bioequivalence between two products. The preferred method for obtaining bioequivalence to a sugar-containing oral transmucosal solid dosage form used in the present invention involves incorporating an ionizing agent into the sugar-free compositions. It is well known that most drugs are weak acids or weak bases and are present in solution in both the non-ionized and ionized forms. It has been found that the non-ionized portion of the drug is usually more lipid soluble and can more readily diffuse across the cell membrane. Ionizing a portion of the drug generally impairs its lipid solubility, and decreases the ability of the drug to penetrate the lipid membrane of the cell or to cross the cell membrane, as a result of the positive or negative charge on the ionized molecules.

Whether a drug exists in the ionized or non-ionized (unionized) form is largely dependent upon its pKa, and correspondingly on the pH of the solution in which it is dispersed. pKa is defined as the negative logarithm (base 10) of the dissociation constant (Ka). pKa may also be defined as the pH at which a given acid or base is 50% ionized and 50% unionized. Using the well-known Henderson-Hasselbalch equation, concentrations of the charged and uncharged species of a drug can easily be calculated, if the pKa and pH are known. From that equation, it is clear that the ionized portion of the drug will be decreased by lowering the pH for weak acid drugs and increasing the pH for drugs that are weak bases. Thus, adding an ionizing agent that maintains a more acidic pH will increase the portion of a basic drug that is present in the ionized form, which may lead to a decrease in the oral transmucosal absorption, and hence, bioavailability. Conversely, adding an ionizing agent that maintains a more basic pH will increase the ionized portion of an acidic drug.

The ionizing agent can be any pharmaceutically acceptable acid or base capable of protonating or deprotonating the ionizable functional groups of the ionizable pharmaceutical agent, in a Brønsted-Lowry sense, or capable of accepting or donating an electron pair, in a Lewis sense. For convenience, the ionizing agents are discussed in terms of Brønsted-Lowry properties, although Lewis acids and bases are also suitable ionizing agents.

Ionizing agents that deprotonate the acidic functional groups of the pharmaceutical agent include pharmaceutically acceptable organic or inorganic bases. Examples of such bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, and the like. Also suitable are bases which are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Preferred cations include sodium, potassium, lithium, magnesium, calcium and ammonium.

Ionizing agents that protonate the basic functional groups of the pharmaceutical agent are pharmaceutically acceptable inorganic or organic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like. Of course, the distinction between inorganic and organic acids is not particularly important, but is provided merely as a convenient and conventional way to classify the acids.

Changes in pH such as those discussed above can preferably be accomplished by incorporating particular buffer systems within the sugar-free composition, in an amount sufficient to maintain a portion of the pharmaceutical agent, upon dissolution of the oral transmucosal solid dosage form, in an ionized state. In this way, the solid dosage form can, to a certain extent, overcome the influence of conditions of the surrounding environment, such as rate of saliva secretion, pH of the saliva, and other factors. Suitable buffer systems may comprise any physiologically acceptable organic and inorganic acids and bases that may be combined in different proportions to produce a buffer having the desired buffering capacity and pH range. The optimum system and pH range will depend on the nature of the drug (acid or base) and compatibility with other formulation ingredients. Buffers may also be selected based upon their ability to impart, enhance or mask certain organoleptic properties in the formulation, such as taste (i.e. salty, sweet, sour, neutral, etc.). A list of exemplary buffers, together with their suitable pH range, is shown in Table 5, below.

TABLE 5

| BUFFER INGREDIENTS | pH RANGE |
|---|---|
| citric acid - sodium hydroxide | 2.5-6.5 |
| citric acid - di-sodium hydrogen phosphate | 2.5-7 |
| citric acid - sodium citrate | 3-6.5 |
| sodium acetate - acetic acid | 3.5-5.5 |
| succinic acid - sodium hydroxide | 3.5-6 |
| potassium dihydrogen phosphate - | 5-8 |
| di-sodium hydrogen phosphate | |
| maleic acid disodium salt - hydrochloric acid | 5.5-6.7 |
| potassium dihydrogen phosphate - | 5.8-8 |
| sodium hydroxide | |
| sodium dihydrogen phosphate - | 5.8-8 |
| di-sodium hydrogen phosphate | |
| imidazole-hydrochloric acid | 6.5-7.5 |
| tris acid maleate - sodium hydroxide | 5-9 |

It should be remembered, however, that solubility of the ionizable pharmaceutical agents is also pH dependent. While increasing the unionized portion makes it easier for the agent to cross the cell membranes, it often decreases the solubility of the drug in aqueous solutions. For example, it is known that the aqueous solubility of fentanyl, an ionizable pharmaceutical agent having a pKa of about 8.4, decreases sharply at a pH above 6, reaching a functional low point for oral mucosal delivery at about pH 8. It has also been shown that the bioavailability of oral transmucosally delivered fentanyl drops to as little as 15% at pH 5, compared to pH 6.5, as a result of the extensive ionization of the drug at that pH. Thus, the actual bioavailability of a fentanyl preparation at a given pH depends on the combination of these two effects; state of ionization and aqueous solubility.

In addition to the pharmaceutical agent, the excipient, and an ionizing compound, such as a buffer, the sugar-free compositions of the present invention may also contain optional ingredients, such as flavorings, sweeteners, flavor enhancers, releasing or lubricating agents, and permeation enhancers. All of these inactive ingredients should preferably be on the GRAS list (“generally regarded as safe”), to assure that they are pharmaceutically acceptable. Alternatively, an inactive ingredient should be self proclaimed GRAS or, at least, acceptable in food.

It will be appreciated that a change of the pH may also change the taste characteristics of the drug. Drugs which are very high in pH typically are very bitter in taste. As the pH drops, the taste becomes less bitter, then salty, and may eventually become sour. Flavorings can more adequately improve the taste characteristics of drugs in the lower pH ranges. As a result, in addition to impacting the bioavailability, buffering pH may also affect the taste characteristics of the composition.

Nonetheless, it may be desirable to add a flavoring agent to the compositions of the present invention. A wide range of flavors are available for preparing good tasting and desirable medications within the scope of the present invention. These may be required in order to mask the unpleasant taste of the drug. Flavorings may be combined, as desired, to produce a particular flavor mix which is compatible with a particular medication. Some of the confectioner's flavorings which may be used in the context of the present invention include artificial vanilla, vanilla cream, mint, berry, cherry, spearmint, grape, coconut, chocolate, menthol, licorice, lemon, and butterscotch. Each of these flavorings is obtainable in a concentrated powder form. Flavoring agents prepared by spray drying are most preferred. Other flavorings known in the confectionary arts may also be acceptable because of the ease of combining the ingredients of the present invention. Any number of flavorings may be combined in any desired ratio in order to produce the specific desired taste characteristics required for any particular application. For example, flavor combinations may be varied in order to be compatible with the flavor characteristics of any specific drug.

In order to produce a desirable color for the end product, artificial colorings may also be added to the composition. The flavorings described above are generally a white powder, as are the other major components. Therefore, additional coloring is necessary if a colored end product is desired. Coloring may also be important as a code to indicate the type and concentration of drug contained within a particular lozenge-on-a-handle. Any type of color known to be “generally regarded as safe” (“GRAS”), and thus generally used in the confectionary trade, or otherwise approved by the appropriate regulatory authority for use in pharmaceutical preparations, may be used to provide coloring to the product.

In order to provide a good tasting medication, it may be necessary to add additional sweeteners to the composition. Since the compositions are sugar-free, an artificial sweetener, such as aspartame, acesulfame K, saccharin, sucralose, altitame, cyclamic acid and its salts, glycerrhizinate, dihydrochalcones, thaumatin, monellin, or any other non-cariogenic, sugar-free sweetner may be used, alone or in combination. For compositions which contain a sugar alcohol based excipient, additional sweeteners may not be necessary, due to the naturally sweet taste of these polyhydric alcohols. Again, it is desired that a sweetener or combination of sweeteners be obtained which is compatible with the pharmaceutical agent and the other components such that a good tasting solid dosage form is produced.

For some applications, it may be desirable to add a flavor enhancer to the composition in order to achieve a good tasting product. Flavor enhancers provide a more pleasant sensation in the patient's mouth during oral transmucosal administration. Flavor enhancers within the scope of the present invention include materials such as ribotide (a nucleotide) and monosodium glutamate (“msg”). Other flavor enhancers are known to those of skill in the art.

In certain medications, it may also be desirable to add a lubricating agent in order to facilitate the manufacturing process. Such agents may also provide a certain amount of waterproofing. The rate of dissolution of the solid dosage form within the patient's mouth may be controlled chemically, as well as physically, for example, through the extent of compression of the composition (where the product is a compressed powder solid dosage form). These lubricating or releasing agents may include substances such as compritol 888, calcium stearate, and sodium stearate. These agents may enhance dissolution or they may inhibit dissolution as necessary.

As discussed above, most drugs are present in solution in both the unionized and ionized forms. Generally lipid soluble or lipophilic drugs diffuse most readily across mucosal membranes. However, it has been found that nonlipophilic drugs may diffuse across mucosal membranes if the mucosal membrane is treated with a permeation enhancer. It has also been found that certain permeability enhancers can significantly enhance the permeability of lipophilic and nonlipophilic drugs.

Typical permeation enhancers may include bile salts such as sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate. Other permeation enhancers such as sodium dodecyl sulfate (“SDS”), dimethyl sulfoxide (“DMSO”), sodium lauryl sulfate, salts and other derivatives of saturated and unsaturated fatty acids, surfactants, bile salt analogs, derivatives of bile salts, or such synthetic permeation enhancers as described in U.S. Pat. No. 4,746,508, the disclosure of which is incorporated herein by reference in its entirety, may also be used. It is generally believed that bile salts are good enhancers for hydrophilic drugs and long chain fatty acids, their salts, derivatives, and analogs are more suitable for lipophilic drugs. DMSO, SDS, and medium chain fatty acids (about C-8 to about C-14) their salts, derivatives, and analogs may work for both hydrophilic and lipophilic drugs.

The permeation enhancer concentration within the dissolvable matrix material may be varied depending on the potency of the enhancer and rate of dissolution of the dissolvable matrix. Other criteria for determining the enhancer concentration include the potency of the drug and the desired lag time. The upper limit for enhancer concentration is set by toxic effect to or irritation limits of the mucosal membrane.

The following is a list of typical enhancers and an exemplary concentration range for each enhancer:

TABLE 6

| | Operational | Preferred |
|---|---|---|
| Enhancer | Concentration | Range |
| sodium cholate | 0.02%-50% | 0.1%-16% |
| sodium dodecyl sulfate | 0.02%-50% | 0.1%-2% |
| sodium deoxycholate | 0.02%-50% | 0.1%-16% |
| taurodeoxycholate | 0.02%-solubility | 0.1%-16% |
| sodium glycocholate | 0.02%-solubility | 0.1%-16% |
| sodium taurocholate | 0.02%-solubility | 0.1%-16% |
| DMSO | 0.02%-solubility | 5%-50% |

The sugar-free solid dosage forms of the present invention may be prepared by a wide variety of methods, including any methods for preparing medicated lozenges, troches, tablets, cough drops, etc., known to those of ordinary skill in the art. Such methods include the following.

Dry powder blending: Dry ingredients (less the lubricant) are combined and mixed for a sufficient time using a suitable low shear diffusion-type mixer. A lubricant is added, followed by a brief remixing, and the final powder blend is compressed into solid dosage units using a tablet press.

Alternatively, dry ingredients (less the lubricant) are combined and mixed sequentially for a sufficient time to achieve uniform dispersion of the ingredients throughout the final powder blend, followed by compression into solid dosage units using a press. An example of sequential mixing is geometric dilution.

Alternatively, the ordering of mixing is somewhere between combining all the ingredients and geometric dilution. For example, the active ingredient is combined and mixed with one other dry ingredient (not the lubricant) for a sufficient time to achieve uniform dispersion of the active ingredient throughout the powder blend, followed by addition of some or all remaining ingredients in an at-one-time or sequential manner. Thus, the active ingredient is pre-blended into one inactive ingredient before addition of all remaining ingredients. The final powder blend is then compressed into solid dosage units using a press.

Wet granulation: The drug, bulking agent and other ingredients, plus a liquid medium, are combined with high shear mixing to form a uniform paste. The paste is dried, and may be ground and sized to form a granulation, which is then formed into solid dosage forms using methods and equipment known to those skilled in the art.

Co-melts or solid dispersions: For heat stable formulations, ingredients with suitable melting points are combined and heated resulting in a solution or a fine dispersion of solids in a liquid medium, that when cooled forms a uniform solid. The resulting solids are often referred to as co-melts, solid solutions and solid dispersions. These intermediate products can then be chopped, ground or compacted into particles of an appropriate size and either compressed, or depending on the ingredients used, molded into solid dosage units, for example by injection molding, as described, for example, in Snipes, U.S. Pat. No. 4,629,621, the disclosure of which is incorporated herein by reference in its entirety, for all purposes.

Tablet making processes other than direct compression of dry powder blends are possible for making sugar-free pharmaceutical compositions. They include, but are not limited to: 1) dry granulation accomplished by slugging or roller compaction or similar processes known to those skilled in the art, 2) low moisture-aided or wet granulation and drying using fluid-bed processing equipment followed by sizing and compression into matrix units using unit processes well known to those skilled in the art, and 3) particle coating of the active ingredient on a carrier ingredient using a fluid-bed process followed by blending with other formulation ingredients and manufacture into solid dosage forms using a tablet press or other suitable means.

Preferred methods for forming solid oral transmucosal dosage forms are described in U.S. Pat. Nos. 4,863,737 and 5,132,114, both to Stanley, et al., which are incorporated herein by reference in their entirety for all purposes.

The aforementioned Stanley patents describe methods for manufacturing compressed powder oral transmucosal solid dosage forms. As used herein, the term “compressed powder oral transmucosal solid dosage form” refers to a solid dosage form in which the ingredients, such as pharmaceutical agent, excipient, ionizing compounds or buffers, and other ingredients have been mixed together in dry form, to produce a powdered blend, that is subsequently compressed to form a solid dosage form suitable for oral transmucosal delivery to a patient. This is to be distinguished from other solid dosage forms, in which the pharmaceutical agent is dispersed in an excipient that is in liquid form, for example having been heated to a temperature above its melting point, or otherwise solubilized, and the liquid blend subsequently allowed to harden into a solid dosage form. Solid dosage forms produced according to this latter method are referred to herein as hard candy oral transmucosal solid dosage forms.

A typical manufacturing method for sugar-containing hard candy oral transmucosal dosage forms consists of the following steps:

1. Solid sucrose is dissolved in liquid dextrose in a heated vessel.

2. A heat-stable drug is added either as a solid, or in solution, and dispersed in the liquefied sugars.

3. The mixture is stirred and heated to drive off excess water, and to bring the mixture to about 150° C.

4. Vacuum is applied to the hot, viscous mixture to remove additional water and bring it to the hard-crack stage (such that the cooked mixture will form a hard, glassy solid when rapidly cooled).

5. Additional ingredients such as buffer, flavor and color are added and mixed into the molten mass.

6. The hot molten material is dispensed and molded into dosage units of suitable shape and size. If a handle is desired, it is generally inserted into the material at this stage.

7. The molded dosage units are allowed to cool and then packaged.

This method is suitable for ingredients that can withstand exposure to high temperatures for the length of time required to complete the process without degradation, and is therefore not suitable for use with thermolabile drugs or excipients.

Whatever manufacturing method is used to prepare the solid dosage form, it is important that the pharmaceutical agent be uniformly dispersed throughout the excipient, so that there is minimal variation between the individual solid dosage forms produced in a given batch. Thus, it is preferred that the actual amount of pharmaceutical agent present in any two solid dosage forms from the same batch is not statistically different. In other words, it is preferred that the ratio of pharmaceutical agent present in two solid dosage forms from the same batch is from about 0.8 to about 1.25, more preferably, from about 0.85 to about 1.15, still more preferably from about 0.9 to about 1.1, and even more preferably, from about 0.95 to about 1.05. It is also preferred that the degree of variation between any two individual solid dosage forms from the same batch meets any official compendial acceptance criteria for solid oral dosage forms.

In preferred solid dosage forms of the present invention, the pharmaceutical agent is preferably present in amounts of from about 0.0005 to about 50% by weight, more preferably in amounts of from about 0.005 to about 10% by weight, and even more preferably, from about 0.005 to about 1% by weight, on a dry weight basis. Assuring a satisfactory level of uniformity may be difficult when a small amount of pharmaceutical agent is dispersed in a very large amount (on a relative basis) of excipient. This is particularly a concern when preparing compressed powder solid dosage forms. One solution to this problem is to use geometric dilution in mixing the various components. Using this method, the two smallest ingredients by weight (as a proportion of the final product) are first mixed together thoroughly. When complete mixing has been obtained between those two components, the next smallest ingredient or ingredients by weight equal to the weight of the previous ingredients is added and mixed thoroughly with the existing mixture. This procedure is repeated until all of the components are added to the mix and mixed thoroughly with all other components. Geometric dilution provides for complete and thorough mixing of all of the components. Using this method, there is little chance for incomplete mixing and uneven distribution of components throughout the mix, assuming the materials do not segregate or demix.

Thus, in accordance with the present invention, there are provided sugar-free oral transmucosal solid dosage forms that are bioequivalent to oral transmucosal solid dosage forms containing one or more sugars, as described in the prior art. In a preferred embodiment, for example, a pharmaceutical composition comprising an oral transmucosal solid dosage form that is substantially free of sugars is prepared using an excipient that comprises a polyhydric alcohol. Preferably, the sugar-free solid dosage form is bioequivalent to a sugar-containing solid dosage form, as described, for example, in U.S. Pat. Nos. 4,671,953; 4,863,737; 5,132,114; 5,288, 497; 5,785,989 and 5,855,908, all to Stanley, et al., prepared in a similar manner, and under similar conditions, but for the substitution of the sugar-free excipient for the sugar-based bulking agents described in the Stanley patents, and the inclusion of an ionizing compound that controls the bioavailability.

A preferred sugar-free oral transmucosal solid dosage form of the present invention comprises fentanyl, a buffer, and an excipient that comprises a polyhydric alcohol or a combination thereof. Other optional ingredients, as discussed previously, may also be present. Fentanyl is preferably included as the citrate salt, in an amount equivalent to from about 50 μg to about 20000 μg of fentanyl free base; preferably from about 50 μg to about 10000 μg of fentanyl free base; preferably from about 50 μg to about 5000 μg of fentanyl free base; preferably from about 50 μg to about 3200 μg of fentanyl free base; more preferably about 100 μg to about 2400 μg of fentanyl free base; more preferably about 200 μg, about 400 μg, about 600 μg, about 800 μg, about 1200 μg, and/or about 1600 μg. Preferably, the buffer is present in an amount sufficient to maintain a portion of the fentanyl, upon dissolution of the composition in saliva, in an ionized state. Thus, the buffer may advantageously act to adjust the rate of transmucosal absorption of the fentanyl, and assist in assuring that the sugar-free fentanyl solid dosage form is bioequivalent to a sugar-containing solid dosage form.

It should be understood that any suitable pharmaceutically acceptable form of the pharmaceutical agent can be used in the compositions of the present invention. For example, fentanyl, if not used as a free base, can also be used as the fentanyl citrate salt, the fentanyl hydrochloride salt, or any additional pharmaceutically acceptable salt known to one skilled in the art. Fentanyl citrate is preferred. With respect to acceptable physical forms of the pharmaceutical agent, it is understood that any suitable pharmaceutically acceptable physical form of the pharmaceutical agent can be used in the compositions of the present invention, for example, powdered, milled, sieved, crystalline, spray dried, lyophilized, or liquid, etc.

Qualitative composition and ingredient ranges for a sugar-free fentanyl citrate solid oral transmucosal delivery form, as contemplated herein, are listed in Table 7. The pharmaceutically acceptable excipient of the composition of Table 7 comprises one or more pharmaceutically acceptable excipients.

TABLE 7

| Ingredient | Content Range (% w/w) |
|---|---|
| Fentanyl citrate | 0.016-0.126 |
| Excipient | 90.88-97.98 |
| Buffer system | 1-5 |
| Flavoring agent | 0.5-2 |
| Lubricant | 0.5-2 |

Preferred buffer systems include citric acid—di-sodium hydrogen phosphate, potassium dihydrogen phosphate—di-sodium hydrogen phosphate, maleic acid disodium salt—hydrochloric acid, potassium dihydrogen phosphate—sodium hydroxide, sodium dihydrogen phosphate—di-sodium hydrogen phosphate, and tris acid maleate—sodium hydroxide. The most preferred buffers are a combination of the sodium or potassium salts of phosphoric acid, or a combination of a mono- or di-salt of phosphoric acid and citric acid. Preferably, the buffer maintains the pH of the composition, upon dissolution in saliva, at a level of from about 5 to about 8. More preferably the buffer maintains the pH of the composition, upon dissolution in saliva, at a level of from about 6 to about 7.4, even more preferably, at a level of from about 6.1 to about 7.0, and still more preferably, at a level of about 6.3 to about 6.6. More preferably the buffer maintains the pH of the composition at about 6.3, about 6.4, about 6.5 or about 6.6. The exact pH needed to establish bioequivalency with a sugar-containing solid oral transmucosal dosage form will depend upon the excipient used, as well as other ingredients in the formulation, and may only be determined by experimental methods known to those skilled in the art.

Certain formulations of the present invention are more preferred. In a more preferred formulation the amount of fentanyl citrate in the pharmaceutical formulation on a percentage weight basis is preferably from about 0.004 to about 0.16% (w/w); more preferably from about 0.008 to about 0.126% (w/w); more preferably from about 0.016 to about 0.126% (w/w); more preferably about 0.016, about 0.032, about 0.048, about 0.064, about 0.096, and/or about 0.126% (w/w). The amount of excipient in the pharmaceutical formulation on a percentage weight basis is preferably from about 75 to about 99% (w/w), more preferably from about 85 to about 99% (w/w), more preferably from about 90 to about 99% (w/w). The amount of buffer in the pharmaceutical formulation on a percentage weight basis is preferably from about 1 to about 5% (w/w), more preferably from about 1.2 to about 4% (w/w), more preferably from about 2 to about 3% (w/w). The amount of optional ingredients in the pharmaceutical formulation on a percentage weight basis is preferably from about 0.0 to about 25% (w/w).

Preferably, the fentanyl sugar-free oral transmucosal solid dosage form is prepared as a compressed powder oral transmucosal solid dosage form, although it could alternatively be prepared as a hard candy oral transmucosal dosage form. Preferably, the solid dosage form has a handle affixed thereto, by any pharmaceutically acceptable means known to those of skill in the art.

The present invention is also directed to methods for the oral transmucosal delivery of a pharmaceutical agent to a patient. These methods comprise the steps of providing a composition comprising a sugar-free oral transmucosal solid dosage form of the present invention, as described previously, and administering the composition to the oral mucosa of a patient. The specific dosages for a given pharmaceutical agent may be readily determined by routine experimentation by those of ordinary skill in the art, without undue effort or inventive contribution.

### EXAMPLES

The invention is further demonstrated in the following Examples. The Examples are for purposes of illustration and are not intended to limit the scope of the present invention.

A list of exemplary sugar-free materials contemplated to be suitable pharmaceutically acceptable excipients useful for preparation of substantially sugar-free oral transmucosal solid dosage forms of the present invention is shown in Table 8. Suitable sugar-free materials are presented as sugar-free excipients or as combinations of compressible sugar-free excipients, bulking agents and binding agents. In the preparation of compressed tablets, compressible sugar-free excipients are preferred.

TABLE 8

| Ingredient/combination | composition |
|---|---|
| Lactitol | Finlac ™ |
| Polydextrose | Litesse ® Ultra ™ |
| Mannitol | Mannogem ™ |
| Mannitol | Mannitol (powder) |
| Mannitol | Pearlitol ® |
| Sorbitol | Sorbidex |
| Sorbitol | Sorbitol |
| Sorbitol | Sorbogem ™ |
| Xylitol | Xylitab-200 |
| Polyalditol | Innovatol |
| Isomalt | Isomalt (compressible) |
| Mannitol and polyalditol | Mannogem ™ and Innovatol (4%, w/w) |
| Mannitol and polyalditol | Mannogem ™ and Innovatol (8%, w/w) |
| Mannitol and polyalditol | Mannogem ™ and Innovatol (16%, w/w) |
| Mannitol and sorbitol | Mannogem ™ and Sorbitol (4%, w/w) |
| Mannitol and sorbitol | Mannogem ™ and Sorbitol (8%, w/w) |
| Mannitol and sorbitol | Mannogem ™ and Sorbitol (16%, w/w) |
| Xylitol and sorbitol | Xylitab-200 and Sorbitol (16%, w/w) |
| Xylitol and sorbitol | Xylitab-200 and Sorbitol (32%, w/w) |
| Xylitol and polyalditol | Xylitab-200 and Innovatol (16%, w/w) |
| Xylitol and polyalditol | Xylitab-200 and Innovatol (32%, w/w) |
| Xylitol and polyalditol | Xylitab-200 and Innovatol (84%, w/w) |
| Xylitol and polyalditol | Xylitab-100 and Innovatol (84%, w/w) |
| Isomalt and polyalditol | Isomalt (compressible) and Innovatol (16%, |
| | w/w) |
| Isomalt and HPC | Isomalt (compressible) and HPC 95 kDa (2%, |
| (hydroxypropylcellulose) | w/w) |
| Isomalt and | Isomalt (compressible) and PEG 4000 (80/20) |
| polyethylene glycol 4000 | |
| Isomalt and PEG 8000 | Isomalt (compressible) and PEG 8000 (80/20) |
| Isomalt and sorbitol | Isomalt (compressible) and Sorbidex (90/10) |
| Isomalt and polydextrose | Isomalt (compressible) and Litesse Ultra |
| | (90/10) |

General Compositions for Sugar-Free Fentanyl Citrate Formulations

The sugar-free fentanyl formulations in the Examples can be made to contain any desired fentanyl dosage strength that may be safely and effectively used to treat the intended painful condition and patient population. In the case of the specific Examples disclosed, the formulations may be made to contain fentanyl (base) dosages ranging from 200 mcg to 1600 mcg per dosage unit. The specific Examples were prepared following the procedure described herein, using the ingredients and ranges listed in Table 9, and tested for bioequivalence using commercially available 800 mcg ACTIQ® brand fentanyl citrate (0.063%, w/w fentanyl citrate).

TABLE 9

| | | Content |
|---|---|---|
| Ingredient | Function | (% w/w) |
| Fentanyl citrate, USP & Ph Eur | Pharmaceutical agent | 0.016-0.126 |
| Isomalt (compressible) | Sugar-free excipient | 76.0 or 86.0 |
| Polyethylene glycol 8000 NF & | Sugar-free excipient/ | 19.0 or 9.5 |
| Ph Eur | binder | |
| Citric acid, anhydrous, | Buffer | 0.0, 0.6 or 0.5 |
| USP & Ph Eur | | |
| Dibasic sodium phosphate, | Buffer | 0.0, 1.4 or 1.5 |
| anhydrous, USP & Ph Eur | | |
| Flavor | Flavoring agent | 0.0 or 1.5 |
| Magnesium stearate, | Lubricant | 1.0 |
| non-bovine, NF & Ph Eur | | |
| Total unit weight (mg) | | 2000 mg |

In Table 9, content (% w/w) percentages may not add exactly to 100 due to rounding.

Additionally, for Examples prepared herein using the general compositions of Table 9 which are unbuffered and/or unflavored the content (% w/w) percentages are 0.0. When an Example is absent buffer or flavor ingredients, the content (% w/w) percentages are compensated by additional compressible isomalt. Ratios of isomalt, PEG 8000 and the buffer components were adjusted in order to make different formulations that are bioequivalent to the commercial 800 mcg ACTIQ® formulation. Isomalt is a compressible grade of a disaccharide polyol mixture made from sucrose by a two-stage process of enzymatic rearrangement followed by catalytic hydrogenation. Isomalt is commercially available in a compressible grade.

General Procedure for Preparation of Examples

The general procedure for all formulations is as follows:

1. The individual ingredients are passed through an appropriate mesh size screen, preferably 20-100 mesh size, to delump or deagglomerate the powder prior to weighing.

2. A preblend powder is made by adding the fentanyl citrate to a portion, preferably less than 20%, of compressible grade of isomalt and blending for a sufficient time to distribute fentanyl citrate into the preblend powder using a diffusion-type mixer.

3. The preblend is transferred to the main blend container containing the other formulation ingredients, less magnesium stearate.

4. The combined ingredients are blended for sufficient time using a diffusion-type mixer to objectively achieve acceptable homogeneity in the main powder blend.

5. Magnesium stearate is added to the main blend and briefly mixed for an additional time using a diffusion-type mixer.

6. The lubricated powder blend is compressed into dosage units using a tablet press.

7. Bulk units are made by inserting a holder into the compressed powder (matrix) units and securing it with food grade glue, examples of which are known to one skilled in the art.

General Procedure for Bioequivalence Testing

Bioequivalence (BE), or relative fentanyl bioavailability (BA), evaluations of sugar-free fentanyl citrate formulations were performed in animals. Animal and human BE tests can be performed using basically the same study design, except that tests are necessarily performed in anesthetized dogs because of the method of administration, while human testing is conducted in awake volunteers. The basic protocol elements for animal BE testing are as follows:

Study Design

The study design used for sugar-free fentanyl citrate formulations is a non-replicated, randomized order of administration, multiple arm crossover design in which one or more sugar-free test formulations is compared to the reference formulation (commercial 800 mcg ACTIQ).

Conduct of the Animal BE Studies

The BE of sugar-free fentanyl citrate Examples were evaluated in a group of 4 or 6 anesthetized purebread Beagle dogs. Each formulation was administered according to a crossover schedule of treatments for all animals. Arterial blood samples were drawn prior to, at the start and at specified times during and after treatment administration and serum samples were analyzed for fentanyl content using LC/MS/MS analysis or methods known to one skilled in the art. Serum fentanyl concentration vs. time profiles were tabulated for each animal and test formulation and the average Cₘₐₓ and AUC were compared with the same averages for the reference product, i.e 800 mcg ACTIQ. The measures of fentanyl Cₘₐₓ and the time of maximum serum concentration (tₘₐₓ) were taken directly from the profiles; AUC was calculated using the trapezoidal rule. The point ratios of the geometric means (the antilog of the log₁₀ transformed means) of the two measures for each test formulation to the reference formulation were compared with the stipulated BE acceptability limit of 80-125%.

PK Criteria for Assessing BE

BE criteria in humans has been established by FDA guidance and are based on maximum drug concentration (Cₘₐₓ), and area under the drug concentration vs. time profile (AUC). BE in humans is statistically defined in terms of the 90% confidence interval (CI) ratios of the mean Cₘₐₓ and AUC of the test formulation(s) to the reference formulation. The acceptability limit for both parameters has been established at 80-125% for most drugs. (See: Guidance for Industry Statistical Approaches to Establishing Bioequivalence, US Department of Health and Human Services, FDA (CDER), January 2001.)

The criteria for assessing BE of sugar-free fentanyl citrate formulations in the dog model are less complex than the criteria used for humans. This is because of the small sample size of the animal studies (typically n=4 or 6) compared to a typical sample size of n=24+in humans. The small sample size of animal studies coupled with the expected interindividual PK variability combine to result in a relatively wide 90% CI for the two PK measures. Hence the BE evaluation in dog studies is based on the point averages of Cₘₐₓ and AUC ratios, not the 90% CI. The tₘₐₓ is not useful for evaluating BE in the animal studies because it is constrained by conditions of drug administration specified in the experimental design.

Test formulations with Cₘₐₓ and AUC ratios that fall between 80-125% of the reference product are considered appropriate candidates for further BE testing in human studies.

### EXAMPLE 1

An unbuffered 2000 mg formulation of sugar-free fentanyl citrate was prepared as disclosed herein with the following composition:

| | % (w/w) | mg/2000 mg |
|---|---|---|
| Ingredients: | | |
| Fentanyl Citrate | 0.063 | 1.2568 |
| Isomalt (compressible)/PEG 8000 (80/20) | 98.9 | 1979 |
| Citric acid | 0 | 0 |
| Dibasic Sodium Phosphate | 0 | 0 |
| Flavor | 0 | 0 |
| Mg Stearate | 1 | 20 |
| **Purity Gum BE | | |
| **Confectioner's sugar | | |
| **Purified water (removed on curing) | | |
| Experimental Results | | |
| Product pH (in SS solution) | 6.99 | |
| Sample size (n) | 4 | |
| Median tₘₐₓ (min) | 15 | |
| Mean Cₘₐₓ ratio | 2.04 | |
| Mean AUC₍₀₋₂₄₀₎ ratio | 1.41 | |

This example formulation was unflavored and unbuffered; pH in solution was determined by the pH 7.0 phosphate buffered saline (SS) used in the pH test.

In Example 1, content (% w/w) percentages may not add exactly to 100, and the sum of ingredients may not add exactly to 2000 mg, due to rounding. Purity Gum BE, Confectioner's sugar, and purified water components were used to make the food-grade (edible) glue used to assemble a holder to the compressed sugar-free fentanyl citrate matrix.

### Example 2

A buffered 2000 mg formulation of sugar-free fentanyl citrate was prepared as disclosed herein with the following composition:

| | |
|---|---|
| % (w/w) | mg/2000 mg |

Ingredients:

| | | |
|---|---|---|
| Fentanyl Citrate | 0.063 | 1.2568 |
| Isomalt (compressible)/ | 95.4 | 1909 |
| PEG 8000 (80/20) | | |
| Citric acid | 0.5 | 10 |
| Dibasic Sodium Phosphate | 1.5 | 30 |
| Berry Flavor | 1.5 | 30 |
| Mg Stearate | 1 | 20 |
| **Purity Gum BE | | |
| **Confectioner's sugar | | |
| **Purified water (removed on curing) | | |
| Experimental Results | | |
| Product pH (in SS solution) | 6.6 | |
| Sample size (n) | 6 | |
| Median tₘₐₓ (min) | 15 | |
| Mean Cₘₐₓ ratio | 1.04 | |
| Mean AUC₍₀₋₂₄₀₎ ratio | 1.10 | |

In Example 2, content (% w/w) percentages may not add exactly to 100, and the sum of ingredients may not add exactly to 2000 mg, due to rounding. Purity Gum BE, Confectioner's sugar, and purified water components were used to make the food-grade (edible) glue used to assemble a holder to the compressed sugar-free fentanyl citrate matrix.

### Example 3

A buffered 2000 mg formulation of sugar-free fentanyl citrate was prepared as disclosed herein with the following composition:

| | |
|---|---|
| % (w/w) | mg/2000 mg |

Ingredients:

| | | |
|---|---|---|
| Fentanyl Citrate | 0.063 | 1.2568 |
| Isomalt (compressible)/ | 95.4 | 1909 |
| PEG 8000 (80/20) | | |
| Citric acid | 0.5 | 10 |
| Dibasic Sodium Phosphate | 1.5 | 30 |
| Peppermint Flavor | 1.5 | 30 |
| Mg Stearate | 1 | 20 |
| **Purity Gum BE | | |
| **Confectioner's sugar | | |
| **Purified water (removed on curing) | | |
| Experimental Results | | |
| Product pH (in SS solution) | 6.6 | |
| Sample size (n) | 6 | |
| Median tₘₐₓ (min) | 15 | |
| Mean Cₘₐₓ ratio | 0.82 | |
| Mean AUC₍₀₋₂₄₀₎ ratio | 0.90 | |

In Example 3, content (% w/w) percentages may not add exactly to 100, and the sum of ingredients may not add exactly to 2000 mg, due to rounding. Purity Gum BE, Confectioner's sugar, and purified water components were used to make the food-grade (edible) glue used to assemble a holder to the compressed sugar-free fentanyl citrate matrix.

### Example 4

A buffered 2000 mg formulation of sugar-free fentanyl citrate was prepared as disclosed herein with the following composition:

| | |
|---|---|
| % (w/w) | mg/2000 mg |

Ingredients:

| | | |
|---|---|---|
| Fentanyl Citrate | 0.063 | 1.2568 |
| Isomalt (compressible)/ | 95.4 | 1909 |
| PEG 8000 (80/20) | | |
| Citric acid | 0.5 | 10 |
| Dibasic Sodium Phosphate | 1.5 | 30 |
| Cherry Flavor | 1.5 | 30 |
| Mg Stearate | 1 | 20 |
| **Purity Gum BE | | |
| **Confectioner's sugar | | |
| **Purified water (removed on curing) | | |
| Experimental Results | | |
| Product pH (in SS solution) | 6.5 | |
| Sample size (n) | 6 | |
| Median tₘₐₓ (min) | 15 | |
| Mean Cₘₐₓ ratio | 0.83 | |
| Mean AUC₍₀₋₂₄₀₎ ratio | 1.03 | |

In Example 4, content (% w/w) percentages may not add exactly to 100, and the sum of ingredients may not add exactly to 2000 mg, due to rounding. Purity Gum BE, Confectioner's sugar, and purified water components were used to make the food-grade (edible) glue used to assemble a holder to the compressed sugar-free fentanyl citrate matrix.

### Example 5

A buffered 2000 mg formulation of sugar-free fentanyl citrate was prepared as disclosed herein with the following composition:

| | |
|---|---|
| % (w/w) | mg/2000 mg |

Ingredients:

| | | |
|---|---|---|
| Fentanyl Citrate | 0.063 | 1.2568 |
| Isomalt (compressible)/ | 95.4 | 1909 |
| PEG 8000 (90/10) | | |
| Citric acid | 0.6 | 12 |
| Dibasic Sodium Phosphate | 1.4 | 28 |
| Berry Flavor | 1.5 | 30 |
| Mg Stearate | 1 | 20 |
| **Purity Gum BE | | |
| **Confectioner's sugar | | |
| **Purified water (removed on curing) | | |
| Experimental Results | | |
| Product pH (in SS solution) | 6.3 | |
| Sample size (n) | 6 | |
| Median tₘₐₓ (min) | 15 | |
| Mean Cₘₐₓ ratio | 0.89 | |
| Mean AUC₍₀₋₂₄₀₎ ratio | 0.87 | |

In Example 5, content (% w/w) percentages may not add exactly to 100, and the sum of ingredients may not add exactly to 2000 mg, due to rounding. Purity Gum BE, Confectioner's sugar, and purified water components were used to make the food-grade (edible) glue used to assemble a holder to the compressed sugar-free fentanyl citrate matrix.

### Example 6

A buffered 2000 mg formulation of sugar-free fentanyl citrate was prepared as disclosed herein with the following composition:

| | |
|---|---|
| % (w/w) | mg/2000 mg |

Ingredients:

| | | |
|---|---|---|
| Fentanyl Citrate | 0.063 | 1.2568 |
| Isomalt (compressible)/ | 95.4 | 1909 |
| PEG 8000 (90/10) | | |
| Citric acid | 0.5 | 10 |
| Dibasic Sodium Phosphate | 1.5 | 30 |
| Berry Flavor | 1.5 | 30 |
| Mg Stearate | 1 | 20 |
| **Purity Gum BE | | |
| **Confectioner's sugar | | |
| **Purified water (removed on curing) | | |
| Experimental Results | | |
| Product pH (in SS solution) | 6.5 | |
| Sample size (n) | 6 | |
| Median tₘₐₓ (min) | 15 | |
| Mean Cₘₐₓ ratio | 1.09 | |
| Mean AUC₍₀₋₂₄₀₎ ratio | 1.30 | |

In Example 1, content (% w/w) percentages may not add exactly to 100, and the sum of ingredients may not add exactly to 2000 mg, due to rounding. Purity Gum BE, Confectioner's sugar, and purified water components were used to make the food-grade (edible) glue used to assemble a holder to the compressed sugar-free fentanyl citrate matrix.

All publications, patents, and patent documents cited herein are incorporated herein by reference, as though individually incorporated by reference. The invention has been described with reference to various specific and preferred embodiments and techniques. It should be understood, however, that many variations and modifications might be made while remaining within the spirit and scope of the invention. When the term “about” is used to modify a numeric value, it means +/−10%.

What is claimed is:

1. A pharmaceutical composition comprising an oral transmucosal solid dosage form comprising an ionizable pharmaceutical agent, a buffer, and a pharmaceutically acceptable excipient, wherein said composition is substantially sugar-free and is bioequivalent to a sugar-containing oral transmucosal solid dosage form, and wherein said buffer is present in an amount sufficient to maintain a portion of said pharmaceutical agent, upon dissolution of said dosage form in saliva, in an ionized state.

2. The composition according to claim 1, wherein said sugar-containing transmucosal solid dosage form contains a sugar selected from the group consisting of glucose, mannose, galactose, ribose, fructose, maltose, sucrose, lactose, and combinations thereof.

3. The composition according to claim 1, wherein said pharmaceutically acceptable excipient comprises a polyhydric alcohol.

4. The composition according to claim 3, wherein said polyhydric alcohol is selected from the group consisting of sorbitol, mannitol, xylitol, erythritol, maltitol, lactitol, isomalt, polyalditol, and combinations thereof.

5. The composition according to claim 4, wherein said polyhydric alcohol is sorbitol.

6. The composition according to claim 4, wherein said polyhydric alcohol is mannitol.

7. The composition according to claim 4, wherein said polyhydric alcohol is xylitol.

8. The composition according to claim 4, wherein said polyhydric alcohol is erythritol.

9. The composition according to claim 4, wherein said polyhydric alcohol is maltitol.

10. The composition according to claim 4, wherein said polyhydric alcohol is lactitol.

11. The composition according to claim 4, wherein said polyhydric alcohol is isomalt.

12. The composition according to claim 4, wherein said polyhydric alcohol is a combination of mannitol and polyalditol; xylitol and polyalditol; isomalt and polyalditol; mannitol and sorbitol; xylitol and sorbitol; and isomalt and sorbitol.

13. The composition according to claim 4, wherein said polyhydric alcohol is a combination of isomalt and polyalditol.

14. The composition according to claim 4, wherein said polyhydric alcohol is a combination of isomalt and sorbitol.

15. The composition according to claim 1, wherein said excipient comprises a polyhydric alcohol and further comprising a binding agent.

16. The composition according to claim 15, wherein said polyhydric alcohol is selected from the group consisting of sorbitol, mannitol, xylitol, maltitol, lactitol, isomalt, polyalditol, and combinations thereof; and said binding agent is selected from the group consisting of polydextrose, cellulosic ether, and polyethylene glycol.

17. The composition according to claim 15, wherein said polyhydric alcohol is isomalt and said binding agent is selected from the group consisting of polydextrose, hydroxypropyl cellulose, and polyethylene glycol, wherein said polyethylene glycol has an average molecular weight of about 3350 to 20,000.

18. The composition according to claim 15, wherein said polyhydric alcohol is isomalt and said binding agent is polyethylene glycol, wherein said polyethylene glycol has an average molecular weight of about 4000 to 8000.

19. The composition according to claim 1, wherein said excipient comprises a non-cariogenic mono-, di-, oligo-, or poly-saccharide.

20. The composition according to one of claims 3-19, wherein said pharmaceutical agent is fentanyl or a pharmaceutically acceptable salt thereof.

21. The composition according to claim 1, wherein said pharmaceutical agent is fentanyl or a pharmaceutically acceptable salt thereof.

22. The composition according to claim 21, wherein said buffer maintains the pH of said dosage form, upon dissolution in saliva, at a level of from about 5 to about 8.

23. The composition according to claim 21, wherein said buffer maintains the pH of said dosage form, upon dissolution in saliva, at a level of from about 6.0 to about 7.4.

24. The composition according to claim 21, wherein said buffer maintains the pH of said dosage form, upon dissolution in saliva, at a level of from about 6.1 to about 7.0.

25. The composition according to claim 21, wherein said buffer maintains the pH of said dosage form, upon dissolution in saliva, at a level of from about 6.3 to about 6.6.

26. The composition according to claim 25, wherein said buffer is a combination of the sodium or potassium salts of phosphoric acid, or a combination of a mono- or di-salt of phosphoric acid and citric acid.

27. The composition according to one of claims 22-26, wherein said pharmaceutically acceptable excipient comprises a polyhydric alcohol.

28. The composition according to one of claims 22-26, wherein said pharmaceutically acceptable excipient comprises a polyhydric alcohol selected from the group consisting of sorbitol, mannitol, xylitol, erythritol, maltitol, lactitol, isomalt, polyalditol, and combinations thereof.

29. The composition according to one of claims 22-26, wherein said pharmaceutically acceptable excipient comprises isomalt.

30. The composition according to claim 1, wherein said buffer is selected from the group consisting of citric acid—sodium hydroxide, citric acid—di-sodium hydrogen phosphate, citric acid—sodium citrate, succinic acid—sodium hydroxide, potassium dihydrogen phosphate—di-sodium hydrogen phosphate, maleic acid disodium salt—hydrochloric acid, potassium dihydrogen phosphate—sodium hydroxide, sodium dihydrogen phosphate—di-sodium hydrogen phosphate, and tris acid maleate—sodium hydroxide.

31. The composition according to claim 30, wherein said buffer is citric acid—di-sodium hydrogen phosphate, potassium dihydrogen phosphate—di-sodium hydrogen phosphate, maleic acid disodium salt—hydrochloric acid, potassium dihydrogen phosphate—sodium hydroxide, sodium dihydrogen phosphate—di-sodium hydrogen phosphate, and tris acid maleate—sodium hydroxide.

32. The composition according to claim 1, wherein said pharmaceutical agent is fentanyl, or a pharmaceutically acceptable salt thereof; said buffer is present in an amount sufficient to maintain the pH of said sugar-free dosage form, upon dissolution in saliva, at a level of from about 6.3 to about 6.6; and wherein said sugar-containing oral transmucosal solid dosage form contains sucrose, glucose, or a combination thereof.

33. The composition according to claim 32, wherein said sugar-containing oral transmucosal solid dosage form contains greater than about 50% by weight sugar, on a dry weight basis.

34. The composition according to claim 33, wherein said sugar-containing oral transmucosal solid dosage form contains greater than about 90% by weight sugar, on a dry weight basis.

35. The composition according to claim 1, wherein said pharmaceutical agent is present in said oral transmucosal solid dosage form in an amount of from about 0.0005 to about 50% by weight, on a dry weight basis.

36. The composition according to claim 35, wherein said pharmaceutical agent is present in said oral transmucosal solid dosage form in an amount of from about 0.005 to about 10% by weight, on a dry weight basis.

37. The composition according to claim 36, wherein said pharmaceutical agent is present in said oral transmucosal solid dosage form in an amount of from about 0.005 to about 1% by weight, on a dry weight basis.

38. The composition according to claim 1 further comprising a handle affixed to said sugar-free oral transmucosal solid dosage form.

39. The composition according to claim 1, wherein said composition is in the form of a compressed powder oral transmucosal solid dosage form.

40. The composition according to claim 1, wherein said composition is in the form of a hard candy oral transmucosal solid dosage form.

41. The composition according to claim 1, wherein said composition is in the form of a hard candy oral transmucosal solid dosage form; further comprising a handle affixed to said hard candy oral transmucosal solid dosage form.

42. The composition according to claim 1, wherein said sugar-containing oral transmucosal solid dosage form contains a buffer.

43. The composition according to claim 1, wherein said sugar-containing oral transmucosal solid dosage form does not contain a buffer.

44. A sugar-free pharmaceutical composition for the oral transmucosal delivery of fentanyl, said composition comprising fentanyl, or a pharmaceutically acceptable salt form thereof, and a pharmaceutically acceptable excipient, wherein said sugar-free composition is in the form of an oral transmucosal solid dosage form, and wherein said oral transmucosal solid dosage form is bioequivalent to a sugar-containing oral transmucosal solid dosage form.

45. The composition according to claim 44, wherein said sugar-containing oral transmucosal solid dosage form contains sucrose, glucose, or a combination thereof.

46. The composition according to claim 44, wherein said sugar-containing oral transmucosal solid dosage form contains greater than about 90% by weight sugar, on a dry weight basis.

47. The composition according to claim 44, wherein the excipient in said sugar-free composition comprises a polyhydric alcohol.

48. The composition according to claim 47, wherein said polyhydric alcohol is selected from the group consisting of sorbitol, manhitol, xylitol, erythritol, maltitol, lactitol, isomalt, polyalditol, and combinations thereof.

49. The composition according to claim 48, wherein said polyhydric alcohol is sorbitol.

50. The composition according to claim 48, wherein said polyhydric alcohol is mannitol.

51. The composition according to claim 48, wherein said polyhydric alcohol is xylitol.

52. The composition according to claim 48, wherein said polyhydric alcohol is erythritol.

53. The composition according to claim 48, wherein said polyhydric alcohol is maltitol.

54. The composition according to claim 48, wherein said polyhydric alcohol is lactitol.

55. The composition according to claim 48, wherein said polyhydric alcohol is isomalt.

56. The composition according to claim 48, wherein said polyhydric alcohol is a combination of mannitol and polyalditol; xylitol and polyalditol; isomalt and polyalditol; mannitol and sorbitol; xylitol and sorbitol; and isomalt and sorbitol.

57. The composition according to claim 48, wherein said polyhydric alcohol is a combination of isomalt and polyalditol.

58. The composition according to claim 48, wherein said polyhydric alcohol is a combination of isomalt and sorbitol.

59. The composition according to claim 44, wherein said excipient comprises a polyhydric alcohol and further comprising a binding agent.

60. The composition according to claim 59, wherein said polyhydric alcohol is selected from the group consisting of sorbitol, mannitol, xylitol, maltitol, lactitol, isomalt, polyalditol, and combinations thereof; and said binding agent is selected from the group consisting of polydextrose, cellulosic ether, and polyethylene glycol.

61. The composition according to claim 59, wherein said polyhydric alcohol is isomalt and said binding agent is selected from the group consisting of polydextrose, hydroxypropyl cellulose, and polyethylene glycol, wherein said polyethylene glycol has an average molecular weight of about 3350 to 20,000.

62. The composition according to claim 59, wherein said polyhydric alcohol is isomalt and said binding agent is polyethylene glycol, wherein said polyethylene glycol has an average molecular weight of about 4000 to 8000.

63. The composition according to claim 44, further comprising a buffer in an amount sufficient to maintain a portion of said fentanyl, or pharmaceutically acceptable salt thereof, upon dissolution of said solid oral transmucosal dosage form in saliva, in an ionized state.

64. The composition according to claim 63, wherein said buffer maintains the pH of said dosage form, upon dissolution in saliva, at a level of from about 6.0 to about 7.4.

65. The composition according to claim 63, wherein said buffer maintains the pH of said dosage form, upon dissolution in saliva, at a level of from about 6.1 to about 7.0.

66. The composition according to claim 63, wherein said buffer maintains the pH of said dosage form, upon dissolution in saliva, at a level of from about 6.3 to about 6.6.

67. The composition according to claim 63, wherein said buffer is selected from the group consisting of citric acid—di-sodium hydrogen phosphate, potassium dihydrogen phosphate—di-sodium hydrogen phosphate, maleic acid disodium salt—hydrochloric acid, potassium dihydrogen phosphate—sodium hydroxide, sodium dihydrogen phosphate—di-sodium hydrogen phosphate, and tris acid maleate—sodium hydroxide.

68. The composition according to claim 63, wherein said buffer is a combination of the sodium or potassium salts of phosphoric acid, or a combination of a mono- or di-salt of phosphoric acid and citric acid.

69. The composition according to one of claims 64-68, wherein the excipient in said sugar-free composition comprises a polyhydric alcohol.

70. The composition according to one of claims 64-68, wherein the excipient in said sugar-free composition comprises a polyhydric alcohol selected from the group consisting of sorbitol, mannitol, xylitol, erythritol, maltitol, lactitol, isomalt, polyalditol, and combinations thereof.

71. The composition according to one of claims 64-68, wherein the excipient in said sugar-free composition is isomalt.

72. The composition according to one of claims 64-68, wherein said excipient comprises a polyhydric alcohol and further comprising a binding agent.

73. The composition according to one of claims 64-68, wherein said polyhydric alcohol is selected from the group consisting of sorbitol, mannitol, xylitol, maltitol, lactitol, isomalt, polyalditol, and combinations thereof; and said binding agent is selected from the group consisting of polydextrose, cellulosic ether, and polyethylene glycol.

74. The composition according to one of claims 64-68, wherein said polyhydric alcohol is isomalt and said binding agent is selected from the group consisting of polydextrose, hydroxypropyl cellulose, and polyethylene glycol, wherein said polyethylene glycol has an average molecular weight of about 3350 to 20,000.

75. The composition according to one of claims 64-68, wherein said polyhydric alcohol is isomalt and said binding agent is polyethylene glycol, wherein said polyethylene glycol has an average molecular weight of about 4000 to 8000.

76. The composition according to claim 44 or 63 further comprising a handle affixed to said sugar-free oral transmucosal solid dosage form.

77. The composition according to claim 44 or 63 wherein said excipient is in the form of a powder, and said composition is in the form of a compressed powder sugar-free oral transmucosal solid dosage form.

78. The composition according to claim 44 or 63 wherein said composition is in the form of a hard candy sugar-free oral transmucosal solid dosage form.

79. The composition according to claim 44, wherein said oral transmucosal solid dosage form contains fentanyl as a fentanyl salt in an amount equivalent to from about 50 μg to about 5000 μg of fentanyl free base.

80. The composition according to claim 44, wherein said oral transmucosal solid dosage form contains fentanyl as a fentanyl salt in an amount equivalent to from about 50 μg to about 3200 μg of fentanyl free base.

81. The composition according to claim 44, wherein said oral transmucosal solid dosage form contains fentanyl as a fentanyl salt in an amount equivalent to from about 50 μg to about 2400 μg of fentanyl free base.

82. The composition according to claim 44, wherein said oral transmucosal solid dosage form contains fentanyl as a fentanyl salt in an amount equivalent to from about 100 μg to about 1600 μg of fentanyl free base.

83. The composition according to claim 82, wherein said fentanyl salt is fentanyl citrate.

84. The composition according to claim 83, wherein said excipient comprises a polyhydric alcohol; and further comprising a buffer.

85. The composition according to claim 84, wherein said excipient comprises isomalt.

86. The composition according to claim 83, wherein said excipient comprises a polyhydric alcohol selected from the group consisting of sorbitol, mannitol, xylitol, maltitol, lactitol, isomalt, polyalditol, and combinations thereof; and further comprising a binding agent selected from the group consisting of polydextrose, cellulosic ether, and polyethylene glycol; and further comprising a buffer.

87. The composition according to claim 86, wherein said excipient comprises a isomalt; and the binding agent comprises polyethylene glycol.

88. The composition according to claim 87 wherein said polyethylene glycol has an average molecular weight of about 3350 to 20,000.

89. The composition according to claim 87 wherein said polyethylene glycol has an average molecular weight of about 4000 to 8000.

90. The composition according to claim 89, wherein said buffer is a combination of the sodium or potassium salts of phosphoric acid, or a combination of a mono- or di-salt of phosphoric acid and citric acid.

91. The composition according to claim 89, wherein said buffer is present in an amount sufficient to maintain the pH of said dosage form, upon dissolution in saliva, at a level of from about 6.1 to about 7.0.

92. The composition according to claim 89, wherein said buffer is present in an amount sufficient to maintain the pH of said dosage form, upon dissolution in saliva, at a level of from about 6.3 to about 6.6.

93. The composition according to claim 92, wherein said oral transmucosal solid dosage form contains fentanyl as fentanyl citrate in an amount equivalent to about 200 μg, about 400 μg, about 600 μg, about 800 μg, about 1200 μg, or about 1600 μg of fentanyl free base.

94. The composition according to claim 63,
wherein
1) the fentanyl is present as fentanyl citrate in an amount equivalent to 0.016 to 0.126 weight %;
2) the excipient is present in an amount equivalent to 90.88 to 97.98 weight %;
3) the buffer is present in an amount equivalent to 1 to 5 weight % and sufficient to maintain the pH of said dosage form, upon dissolution in saliva, at a level of from about 6.3 to about 6.6; and
further comprising 4) a lubricant present in an amount equivalent to 0.5 to 2 weight %.

95. The composition according to claim 63,
wherein
1) the fentanyl is present as fentanyl citrate in an amount equivalent to 0.016 to 0.126 weight %;
2) the excipient is present as isomalt in an amount equivalent to 76.0 to 86.0 weight %;
3) the buffer is present as citric acid in an amount equivalent to 0.5 to 0.6 weight % combined with dibasic sodium phosphate in an amount equivalent to 1.4 to 1.5 weight % and sufficient to maintain the pH of said dosage form, upon dissolution in saliva, at a level of from about 6.3 to about 6.6;
further comprising 4) a binding agent present as polyethylene glycol 8000 in an amount equivalent to 9.5 to 19.0 weight % and
further comprising 5) a lubricant present as magnesium stearate in an amount equivalent to 1.0 weight %.

96. A method for the oral transmucosal delivery of a pharmaceutical agent in a sugar-free dosage form to a patient, comprising providing a composition according to claim 1, and administering an effective amount of said composition to the oral mucosa of said patient, and delivering said pharmaceutical agent by absorption through a patient's oral mucosal tissue.

97. A method for the oral transmucosal delivery of fentanyl in a sugar-free dosage form to a patient, comprising providing a composition according to claim 44, and administering an effective amount of said composition to the oral mucosa of said patient, and delivering said pharmaceutical agent by absorption through a patient's oral mucosal tissue.

98. A method of treating pain which comprises introducing into the oral cavity of a patient a therapeutically effective amount of a sugar-free oral transmucosal solid dosage form according to claim 1 wherein the ionizable pharmaceutical agent is fentanyl, or a pharmaceutically acceptable salt form thereof.

99. The method according to claim 98 in which the pain is breakthrough pain.

100. The method according to claim 98 in which the pain is chronic pain.

101. The method according to claim 98 in which the pain is migraine pain.

102. A method of treating pain which comprises introducing into the oral cavity of a patient a therapeutically effective amount of a sugar-free oral transmucosal solid dosage form according to claim 44.

103. The method according to claim 102 in which the pain is breakthrough pain.

104. The method according to claim 102 in which the pain is chronic pain.

105. The method according to claim 102 in which the pain is migraine pain.
